(19) United States
(12) Patent Application Publication
(10) Pub. No.: US 2023/0159524 A1
(43) Pub. Date: May 25, 2023

(54) METHODS FOR SYNTHESIZING N-(PHENYLSULFONYL)BENZAMIDE COMPOUNDS AND INTERMEDIATES THEREOF

(71) Applicants: ASCENTAGE PHARMA (SUZHOU) CO., LTD., Jiangsu (CN); ASCENTAGE PHARMA GROUP CORP LIMITED, Hong Kong (CN)

(72) Inventors: Jianfeng WEN, Jiangsu (CN); Jianyong CHEN, Jiangsu (CN); Yunlong ZHOU, Jiangsu (CN); Jianpeng FENG, Jiangsu (CN); Ming GUO, Jiangsu (CN); Tianzhu WU, Jiangsu (CN); Minmin CAI, Jiangsu (CN); Yu JING, Jiangsu (CN); Lingling JIAO, Jiangsu (CN)

(73) Assignees: ASCENTAGE PHARMA (SUZHOU) CO., LTD., Jiangsu (CN); ASCENTAGE PHARMA GROUP CORP LIMITED, Hong Kong (CN)

(21) Appl. No.: 17/921,257

(22) PCT Filed: Jul. 1, 2021
(86) PCT No.: PCT/CN2021/103928

§ 371 Date: Oct. 25, 2022

(30) Foreign Application Priority Data
Jul. 1, 2020 (CN) 202010626349.1

(51) Int. Cl. C07D 471/04; C07D 295/073; C07D 319/12; C07C 63/10; C07C 47/457; B01J 31/24

(52) U.S. Cl. CPC C07D 471/04; C07D 295/073; C07D 319/12; C07C 63/10; C07C 47/457; B01J 31/24; C07C 2602/50

(57) ABSTRACT

Disclosed is a method for synthesizing N-(phenylsulfonyl)benzamide compound and intermediate thereof. The method comprises a method for synthesizing a compound 1, comprising conducting a Buchwald-Hartwig coupling reaction as shown below with compound A and compound B in a solvent and in the presence of a base and a palladium catalyst to obtain the compound 1; wherein R is C₁-C₈ alkyl. The present disclosure synthesizes three intermediate compounds required by the target compound and their preparation methods for the first time. Using the method disclosed in the present disclosure to synthesize the target compound 3 has the advantages of high yield, good purity, easy-to-obtain reaction raw materials, suitable for industrial production.

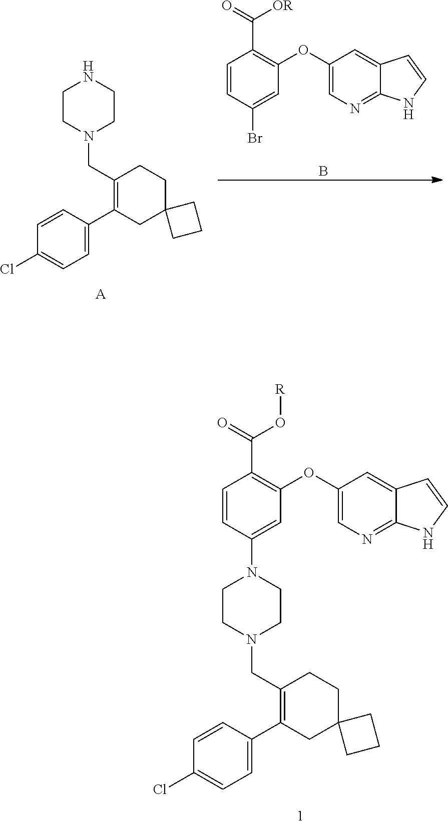

### TECHNICAL FIELD

The present disclosure relates to the fields of medicinal chemistry. Specifically, it relates to methods for synthesizing N-(phenylsulfonyl)benzamide compounds and intermediates thereof.

### BACKGROUND

Apoptosis is a process of programmed cell death and an essential biological process of tissue homeostasis. In mammals, it has been demonstrated that early embryonic development can be modulated by apoptosis. In the later stages of life, cell death is a default mechanism by which potentially dangerous cells, such as those carrying cancer defects, are removed. Several apoptotic pathways are known. One of the most important apoptotic pathways relates to the Bcl-2 protein family, which is a key regulator of the apoptotic mitochondria (also known as ‘intrinsic’) pathway.

Dysregulated apoptosis pathways involve the pathology of many important diseases, such as neurodegenerative disorders (up-regulated apoptosis), e.g., Alzheimer's disease; and proliferative diseases (down-regulated apoptosis), e.g., cancers, autoimmune diseases and prothrombotic disorders. Down-regulated apoptosis (more specifically, the Bcl-2 protein family) can participate in the onset of cancerous malignancies. Studies have shown that, for example, anti-apoptotic proteins Bcl-2 and Bcl-xL overexpress down-regulated apoptosis in many cancer cells types (more specifically, the Bcl-2 protein family), which can participate in the onset of cancerous malignancies. Studies have shown that, for example, anti-apoptotic proteins Bcl-2 and Bcl-xL are overexpressed in many cancer cells types. The survival of cancer cells is attributed to the dysregulation of the apoptotic pathway caused by the overexpression of one or more anti-apoptotic Bcl-2 protein family members. In view of the important role of the Bcl-2 protein family in the regulation of cancer cells and normal cells (i.e. non-cancer cells) apoptosis, as well as the recognized inter-cell type variability of Bcl-2 family protein expression, thus, it is beneficial as small molecule inhibitors that selectively target and preferably bind to one type or a part of anti-apoptotic Bcl-2 proteins, for example, bind to anti-apoptotic Bcl-2 family members that are overexpressed in a certain cancer type.

It has been reported that N-(phenylsulfonyl)benzamide compounds are effective Bcl-2 inhibitors, and one of which has the following structural formula:

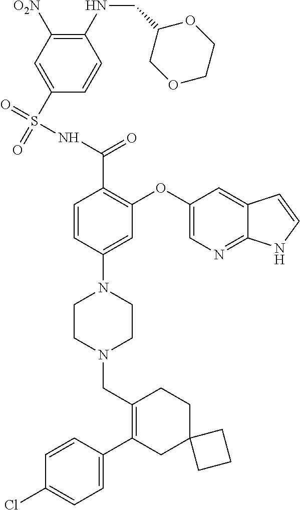

At present, there are few reports on the methods for preparing N-(phenylsulfonyl)benzamide compounds in the prior art, so it is urgent to prepare N-(phenylsulfonyl)benzamide compounds efficiently and economically.

### CONTENT OF THE PRESENT INVENTION

In the first aspect, the present disclosure provides a method for synthesizing compound 1, which comprises conducting a Buchwald-Hartwig coupling reaction as shown below with compound A and compound B in a solvent and in the presence of a base and a palladium catalyst to obtain the compound 1;

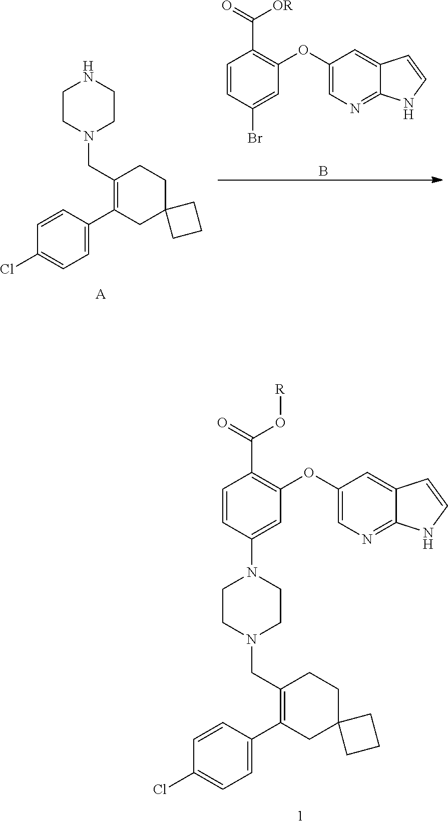

wherein R is C₁-C₈ alkyl.

In some embodiments, in the method for synthesizing the compound 1, the palladium catalyst can be a conventional palladium catalyst for such reactions in the art, for example palladium acetate, [1,1′-bis(diphenylphosphino)ferrocene]dichloro palladium(II), tetrakis(triphenylphosphine)palladium, bis(triphenylphosphine) palladium(II) chloride, palladium on carbon, palladium hydroxide, [1,3-bis(2,6-diisopropylphenyl)imidazol-2(3H)-ylidene](3-chloro-1-pyridyl)palladium(IV) chloride, tris(dibenzylideneacetone) dipalladium, bis(di-tert-butyl(4-(dimethylaminophenyl)phosphine)dichloropalladium(II), or a mixture thereof, preferably bis(di-tert-butyl(4-(dimethylaminophenyl)phosphine)dichloropalladium(II). A molar ratio of the compound A to the palladium catalyst can be 1: (0.01-0.5), preferably 1:(0.05-0.2).

In some embodiments, in the method for synthesizing the compound 1, a molar ratio of the compound A to the compound B can be 1:(1-3), preferably 1:(1-1.5).

In some embodiments, in the method for synthesizing the compound 1, the base can be a conventional base for such reactions in the art, such as an inorganic base, an organic base, or a mixture thereof. The inorganic base can be an alkali metal hydroxide, an alkali metal carbonate, an alkali metal phosphate, an alkali metal bicarbonate or a mixture thereof, such as sodium hydroxide, potassium hydroxide, potassium carbonate, sodium carbonate, potassium phosphate, lithium carbonate, cesium carbonate or a mixture thereof. The organic base can be R^{m1}OM1, (M2)N(Rⁿ¹Rⁿ²) or a mixture thereof, M1 and M2 are independently alkali metal; Rⁿ¹ and Rⁿ² are independently C₁-C₄ alkyl or —Si(R^{s1}R^{s2}R^{s3}), R^{m1}, R^{s1}, R^{s2} and R^{S3} are independently C₁-C₄ alkyl; for example, potassium tert-butoxide, sodium tert-butoxide, n-butyllithium, KHMDS, NaHMDS, LDA, potassium tert-pentoxide, sodium tert-pentoxide or a mixture thereof. A molar ratio of the compound A to the base can be 1:(3-10), preferably 1:(4.5-8).

In some embodiments, in the method for synthesizing the compound 1, the solvent can be a conventional solvent for such reactions in the art, for example, a chlorinated alkane solvent, an aromatic hydrocarbon solvent, an ether solvent or a mixture thereof, preferably a mixed solvent of two or more solvents, such as a mixed solvent of the aromatic hydrocarbon solvent and the ether solvent. An amount of each solvent in the mixed solvent may not be specifically limited. A mass ratio of the aromatic hydrocarbon solvent and the ether solvent can be 1:1-10:1, preferably 1:1-5:1, more preferably 1:2. The chlorinated alkane solvent can be dichloromethane, chloroform, 1,2-dichloroethane or a mixture thereof. The aromatic hydrocarbon solvent can be benzene, toluene, xylene, chlorobenzene or a mixture thereof. The ether solvent can be diethyl ether, tetrahydrofuran, 2-methyltetrahydrofuran, 1,4-dioxane, methyl tert-butyl ether, ethylene glycol dimethyl ether or a mixture thereof. A mass ratio of the solvent to the compound A can be 1:1-10:1, for example, 5:1-10:1.

In some embodiments, in the method for synthesizing the compound 1, the temperature of the Buchwald Hartwig coupling reaction can be a conventional temperature for such reactions in the art, such as 40-100° C., preferably 40-70° C., more preferably 55-70° C.

In some embodiments, in the method for synthesizing the compound 1, the progress of the Buchwald Hartwig coupling reaction can be detected by conventional methods in the art (e.g., HPLC, GC, TLC or NMR, etc.), and the disappearance of compound A detected by HPLC is generally regarded as the completion of the reaction. The time of the Buchwald Hartwig coupling reaction can be 1-5 hours, preferably 1-2 hours.

In some embodiments, in the method for synthesizing the compound 1, the Buchwald-Hartwig coupling reaction is preferably conducted under the protection of a gas. The gas in the gas protection does not participate in the reaction, and can be nitrogen, helium or argon.

In some embodiments, the Buchwald-Hartwig coupling reaction can be conducted in the presence of a ligand or in the absence of a ligand. When the Buchwald-Hartwig coupling reaction is conducted in the presence of the ligand, then the ligand can be a conventional ligand of a palladium catalyst, such as a phosphine ligand. The phosphine ligand can be selected from monodentate phosphine ligands such as triphenylphosphine (CAS:603-35-0), triphenylphosphine-3,3′,3″-trisulfonic acid trisodium salt (CAS:63995-70-0), tris(o-methylphenyl)phosphine (CAS:6163-58-2), 1,2,3,4,5-pentaphenyl-1′-(di-tertbutylphosphino)ferrocene (CAS:312959-24-3), or a mixture thereof. The phosphine ligand can also be selected from polydentate phosphine ligands such as 1,1′-binaphthyl-2.2′-diphemyl phosphine (CAS:98327-87-8), bis(2-diphenylphosphinophenyl)ether (CAS:166330-10-5), 1,6-bis(diphenylphosphino) hexane (CAS:19845-69-3), 1,1′-bis(diphenylphosphino)ferrocene (CAS:12150-46-8), 4,6-bis(diphenylphosphino)phenoxazine (CAS:261733-18-0), or a mixture thereof.

In some embodiments, in the method for synthesizing the compound 1, the phosphine ligand can be selected from (4-(N,N-dimethylamino)phenyl)di-tert-butyl phosphine (CAS: 932710-63-9), tris(2-furanyl)phosphine (CAS: 5518-52-5), 1,3-di-tert-butyl-1,3,2-diazaphospholidine 2-oxide (CAS: 854929-38-7), 1-[2-(di-tert-butylphosphanyl)phenyl]-3,5-diphenyl-1H-pyrazole (CAS: 628333-86-8), 4,5-bis(diphenylphosphino)-9,9-dimethylxanthene (CAS: 161265-03-8), 2-dicyclohexylphosphino-2′,6′-di-i-propoxy-1,1′-biphenyl (CAS: 787618-22-8) or a mixture thereof. When the Buchwald-Hartwig coupling reaction is conducted in the presence of a ligand, a molar ratio of the compound A to the ligand can be 1:(0.01-0.5), preferably 1:(0.05-0.2).

In some embodiments, the method for synthesizing the compound 1 preferably comprises under the protection of the gas, mixing a mixture of the compound A, the compound B and the solvent with the catalyst and the base to conduct the Buchwald-Hartwig coupling reaction.

In some embodiments, a post-treatment in the method for synthesizing the compound 1 can be a conventional post-treatment for such reactions in the art. For the post-treatment, for example, an amino acid compound can be used to remove the residual metallic palladium in the reaction after the reaction is completed. The amino acid compound can be, for example, cysteine, N-acetyl-L-cysteine, ethylenediaminetetraacetic acid, sodium ethylenediaminetetraacetate, dithiocarbamate compounds or a mixture thereof; for example, cysteine, N-acetyl-L-cysteine or a mixture thereof; for example, N-acetyl-L-cysteine. An amount of the amino acid compound may not be specifically limited, as long as the palladium remaining in the reaction solution after the reaction can be completely removed.

In some embodiments, the post-treatment in the method for synthesizing the compound 1 preferably comprises adjusting the pH of the reaction solution after the completion of Buchwald-Hartwig coupling reaction to 4-5 with an acid (such as a dilute hydrochloric acid aqueous solution), extracting with an ester solvent (such as ethyl acetate), and treating the organic phase with an amino acid compound (the amino acid compound is as defined in the present disclosure, preferably N-acetyl-L-cysteine sodium bicarbonate aqueous solution, wherein, the mass ratio of N-acetyl-L-cysteine:sodium bicarbonate:water=1:1.1:12.8), washing the obtained organic phase with saturated brine, drying (such as anhydrous sodium sulfate or anhydrous magnesium sulfate), and concentrating (concentrating under reduced pressure) to obtain the compound 1.

In some embodiments, in the method for synthesizing the compound 1, a salt of the compound A can also be used to conduct the Buchwald-Hartwig coupling reaction. The salt of the compound A can be a salt formed by the compound A and an acid. The acid can be a conventional inorganic acid or organic acid in the art. The inorganic acid can be hydrochloric acid, sulfuric acid or phosphoric acid, preferably hydrochloric acid. The organic acid can be trifluoroacetic acid. When a salt of the compound A is used to conduct the Buchwald-Hartwig coupling reaction, then the salt of the compound A can be dissociated to the compound A by conventional methods in the art and then participate in the reaction. In the present disclosure, a method for dissociating comprises dissociating the salt of the compound A in a solvent and in the presence of a base to obtain the compound A;

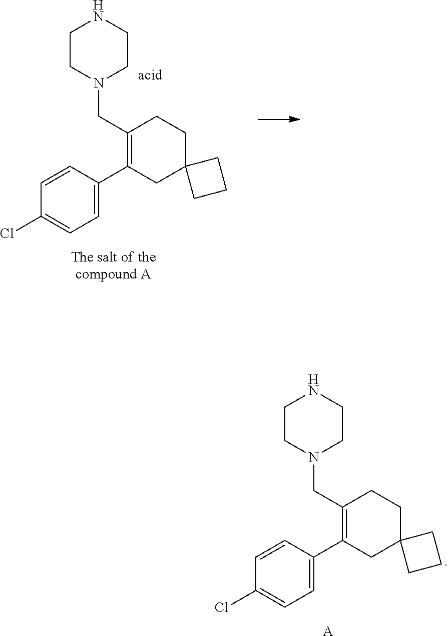

In the method for dissociating the salt of the compound A, the base can be a conventional base for such reactions in the art, such as an alkali metal carbonate, an alkali metal bicarbonate or a mixture thereof, e.g., sodium carbonate, sodium bicarbonate or a mixture thereof. An amount of the base is generally to make the pH value in the reaction solution between 8 and 9. It can be understood that the dissociation of the salt of compound A to the compound A is conducted under the condition of pH 8-9.

In the method for dissociating the salt of the compound A, the solvent can be a conventional solvent for such reactions in the art, for example, a mixed solvent of water and an organic solvent. The organic solvent is preferably an organic solvent that can be layered with water and has good solubility for the compound A, such as a chlorinated alkane solvent, an aromatic hydrocarbon solvent or a mixture thereof. The chlorinated alkane solvent can be dichloromethane, chloroform, 1,2-dichloroethane or a mixture thereof. The aromatic hydrocarbon solvent can be benzene, toluene, xylene, chlorobenzene or a mixture thereof. In the mixed solvent, a mass ratio of water to the organic solvent can be 1:1-1:20, preferably 1:1-1:16, for example 1:1-1:10, e.g., 1:1-1:8. An amount of the solvent may not be specifically limited, and the mass ratio of the solvent to the salt of the compound A can be 1:1-1:20, preferably 1:1-1:16, for example 1:1-1:10, e.g., 1:1-1:8.

In the method for synthesizing the compound 1, the method can further comprise a method for synthesizing the salt of the compound A, which preferably comprises conducting a reaction as shown below with a compound A-7 in a solvent and in the presence of an acid to obtain the salt of the compound A;

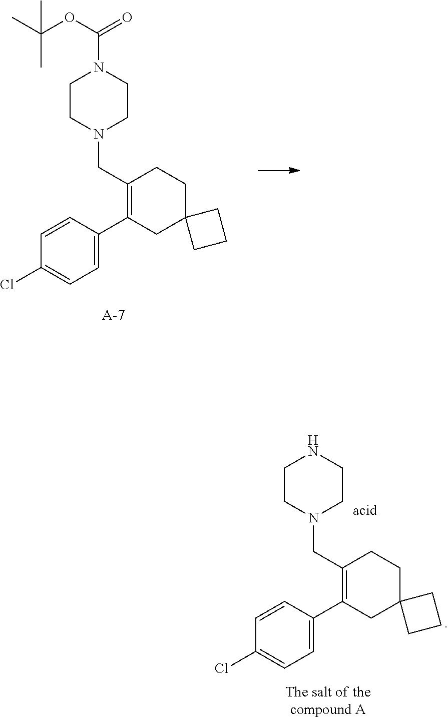 [SMILES: CC(C)(C)OC(=O)N1CCN(CC2=C(c3ccc(Cl)cc3)CC3(CCC3)CC2)CC1 ; "A-7" ; Clc1ccc(C2=C(CN3CCNCC3)CCC3(CCC3)C2)cc1 ; "The salt of thecompound A"]

In the method for synthesizing the salt of the compound A, the acid can be a conventional acid in the art, for example, an inorganic acid or an organic acid. The inorganic acid can be hydrochloric acid, sulfuric acid or phosphoric acid. The hydrochloric acid is preferably concentrated hydrochloric acid with a mass fraction of 36%. The organic acid can be trifluoroacetic acid. An amount of the acid can be a conventional amount for such reactions in the art. A mass ratio of the compound A-7 to the acid can be 1:(1-10), preferably 1:(5-10).

In the method for synthesizing the salt of the compound A, the solvent can be a conventional solvent for such reactions in the art, for example, water, a C₁-C₆ alcohol solvent, a chlorinated alkane solvent, an ether solvent, an ester solvent or a mixture thereof. The C₁-C₆ alcohol solvent can be methanol, ethanol, isopropanol, tert-butanol, n-butanol or a mixture thereof. The chlorinated alkane solvent can be dichloromethane, chloroform, 1,2-dichloroethane or a mixture thereof. The ether solvent can be diethyl ether, tetrahydrofuran, 2-methyltetrahydrofuran, 1,4-dioxane, methyl tert-butyl ether, ethylene glycol dimethyl ether or a mixture thereof. The ester solvent is, for example, ethyl acetate, isopropyl acetate or a mixture thereof. An amount of the solvent may not be specifically limited as long as it does not affect the progress of the reaction. Amass ratio of the compound A-7 to the solvent can be 1:(1-10), preferably 1:(5-10).

In the method for synthesizing the salt of the compound A, the reaction temperature can be 30-100° C., preferably 30-70° C., for example 60-70° C.

In the method for synthesizing the salt of the compound A, the progress of the reaction can be detected by conventional methods in the art (e.g., TLC, GC, HPLC or NMR, etc.), and the disappearance of compound A-7 detected by HPLC is generally regarded as the completion of the reaction. The time of the reaction can be 10-20 hours, preferably 15-20 hours.

The method for synthesizing the salt of the compound A preferably comprises mixing a mixed solution of the compound A-7 and the solvent with the acid (the mixing temperature is preferably room temperature) to conduct the reaction.

A post-treatment method in the method for synthesizing the salt of the compound A can be a conventional post-treatment method for such reactions in the art, the post-treatment preferably comprises stirring the reaction solution after the completion of the reaction at 40-50° C. for 1-2 hours, and then stirring at 0-10° C. for 1-2 hours, filtering, optionally washing the filter cake (preferably washing with an alcohol solvent, such as isopropanol) to obtain a crude product; recrystallizing the crude product to obtain the salt of the compound A.

The solvent of the recrystallization is preferably a mixed solvent of an alcohol solvent and water (for example, a mixed solvent of isopropanol and water, a mass ratio of isopropanol and water is preferably 10:1-20:1, more preferably 10:1-15:1). Amass ratio of the solvent of the recrystallization to the crude product can be 3:1-10:1, preferably 5:1-8:1. The temperature of the recrystallization is preferably the reflux temperature of the alcohol solvent under normal pressure. The time of the recrystallization is preferably 2-3 hours. After the recrystallization is completed, optionally, the solution of the recrystallization is stirred at 40-50° C. for 1-2 hours, then stirred at 0-10° C. for 1-2 hours, filtered, and the solid is dried (for example, drying under vacuum at 45-50° C. for 11 hours) to obtain the salt of the compound A.

The method for synthesizing the salt of the compound A can further comprise a method for synthesizing the compound A-7, which preferably comprises conducting a Borch reduction as shown below with compound A-6 and 1-Boc-piperazine in a solvent and in the presence of a reducing agent to obtain the compound A-7;

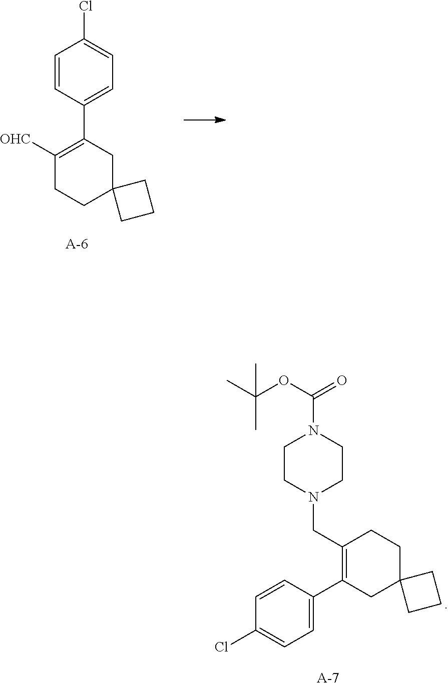

In the method for synthesizing the compound A-7, the reducing agent can be a conventional reducing agent for such reactions in the art, for example a metal borohydride, such as NaCNBH₃, NaBH(OAc)₃, NaBH₄ or a mixture thereof, e.g., NaBH(OAc)₃. An amount of the reducing agent can be a conventional amount for such reactions in the art. A molar ratio of the compound A-6 to the reducing agent can be 1:(1-10), preferably 1:(3-10).

In the method for synthesizing the compound A-7, the solvent can be a conventional solvent for such reactions in the art, for example, a chlorinated alkane solvent, an ether solvent, a nitrile solvent, an ester solvent or a mixture thereof. The chlorinated alkane solvent can be dichloromethane, chloroform, 1,2-dichloroethane or a mixture thereof. The ether solvent can be diethyl ether, tetrahydrofuran, 2-methyltetrahydrofuran, methyl tert-butyl ether or a mixture thereof. The nitrile solvent can be acetonitrile. The ester solvent can be ethyl acetate, isopropyl acetate or a mixture thereof. An amount of the solvent may not be specifically limited as long as it does not affect the progress of the reaction. Amass ratio of the compound A-6 to the solvent can be 1:(1-25), preferably 1:(1-15).

In the method for synthesizing the compound A-7, an amount of the compound A-6 and 1-Boc-piperazine can be a conventional amount for such reactions in the art. A molar ratio of the compound A-6 to 1-Boc-piperazine can be 1:(1-5), preferably 1:(1.5-4).

In the method for synthesizing the compound A-7, the temperature of the Borch reduction can be a conventional temperature for such reactions in the art, for example, room temperature to 50° C., preferably 25-35° C.

In the method for synthesizing the compound A-7, a progress of the Borch reduction can be detected by conventional methods in the art (e.g., TLC, GC, HPLC or NMR, etc.), and the disappearance of compound A-6 detected by HPLC is generally regarded as the completion of the reaction. The time of the Borch reduction can be 1-5 hours, preferably 2-3 hours.

The method for synthesizing the compound A-7 is preferably conducted under the protection of a gas. The gas in the gas protection does not participate in the reaction, and can be nitrogen, helium or argon.

The method for synthesizing the compound A-7 preferably comprises adding the reducing agent to a mixed solution of the 1-Boc-piperazine, the compound A-6 and the solvent under the protection of gas to conduct the reaction, more preferably comprises adding 1-Boc-piperazine, the compound A-6 and the solvent successively, then adding the reducing agent under the protection of gas to conduct the reaction.

A post-treatment in the method for synthesizing the compound A-7 can be a conventional post-treatment for such reactions in the art. The present disclosure preferably comprises mixing the mixed solution after the completion of the reaction with water at −5-5° C., then adjusting the pH of the mixed solution to 7-8 (for example, the pH is adjusted with 20% NaOH aqueous solution), mixing the obtained organic phase with activated carbon, refluxing, filtering while hot, concentrating the filtrate under reduced pressure to dryness, recrystallizing (for example, recrystallizing with acetonitrile), filtering, and drying to obtain compound A-7.

The method for synthesizing the compound A-7 can further comprise a method for synthesizing compound A-6, which preferably comprises conducting a coupling reaction as shown below with compound A-5 and 4-chloro phenylboronic acid in a solvent and in the presence of a palladium catalyst and a base to obtain the compound A-6 under the protection of a gas;

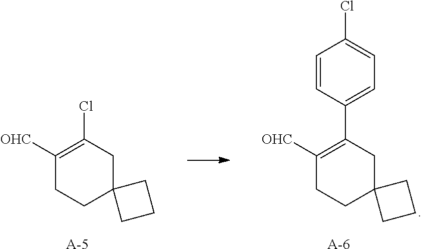

In the method for synthesizing the compound A-6, the gas in the gas protection does not participate in the reaction, such as nitrogen, helium or argon.

In the method for synthesizing the compound A-6, the palladium catalyst can be a conventional palladium catalyst for such reactions in the art, for example palladium acetate, [1,1′-bis(diphenylphosphino)ferrocene]dichloro palladium(II), tetrakis (triphenylphosphine)palladium, bis(triphenylphosphine) palladium(II) chloride, palladium on carbon, palladium hydroxide, [1,3-bis(2,6-diisopropylphenyl)imidazol-2(3H)-ylidene](3-chloro-1-pyridyl)palladium(IV) chloride, tris(dibenzylideneacetone) dipalladium, bis(di-tert-butyl(4-(dimethylaminophenyl)phosphine)dichloropalladium(II), or a mixture thereof. An amount of the palladium catalyst can be a conventional amount for such reactions in the art. A molar ratio of the compound A-5 to the palladium catalyst can be 1:(0.001-0.05), preferably 1:(0.001-0.03).

In the method for synthesizing the compound A-6, an amount of the compound A-5 and 4-chlorophenylboric acid can be a conventional amount in the art. A molar ratio of the compound A-5 to 4-chlorophenylboric acid can be 1:(0.8-2.5), preferably 1:(0.8-1.5).

In the method for synthesizing the compound A-6, the base can be an inorganic base. The inorganic base can be an alkali metal carbonate, such as cesium carbonate, potassium carbonate, sodium carbonate, lithium carbonate or a mixture thereof. The alkali activity is Cs₂CO₃>K₂CO₃>Na₂CO₃>Li₂CO₃. An amount of the base can be a conventional amount for such reactions in the art. A molar ratio of the compound A-5 to the base can be 1:(1-5), preferably 1:(2-5).

In the method for synthesizing the compound A-6, the solvent can be a conventional solvent for such reactions in the art, for example, water, a C₁-C₆ alcohol solvent, an ether solvent or a mixture thereof, preferably a mixture of two or more solvents, such as a mixed solvent of the ether solvent, the alcohol solvent and water. An amount of each solvent in the mixed solvent may not be specifically limited. Optionally, a mass ratio of the ether solvent to water and the alcohol solvent is (1-50):(1-50):1, for example (1-10):(1-10):1. The C₁-C₆ alcohol solvent can be methanol, ethanol, isopropanol, tert-butanol, n-butanol or a mixture thereof. The ether solvent can be diethyl ether, tetrahydrofuran, 2-methyltetrahydrofuran, 1,4-dioxane, methyl tert-butyl ether, ethylene glycol dimethyl ether or a mixture thereof. A mass ratio of the solvent to the compound A-5 can be 1:1-50:1, for example, 5:1-20:1.

In the method for synthesizing the compound A-6, the temperature of the coupling reaction can be a conventional temperature for such reactions in the art, for example, 30-70° C., preferably 40-60° C., more preferably 40-50° C.

In some embodiments, in the method for synthesizing the compound A-6, a progress of the coupling reaction can be detected by conventional methods in the art (e.g., HPLC, GC, TLC or NMR, etc.), and the disappearance of compound A-5 detected by HPLC is generally regarded as the completion of the reaction. The time of the coupling reaction can be 1-5 hours, preferably 1-2 hours.

The method for synthesizing the compound A-6 preferably comprises mixing a mixture of the compound A-5 and the solvent with the base under the protection of the gas, then adding palladium catalyst, then adding (temperature such as 30-70° C., preferably 40-60° C., more preferably 40-50° C.) a mixed solution of 4-chlorophenylboronic acid and the solvent to conduct the coupling reaction.

The method for synthesizing the compound A-6 can also be conducted in the presence of a fluorine-containing additive. The fluorine-containing additive can generate fluoride ions in the reaction solution. The generated fluoride ion can promote the combination of the borate intermediate and the palladium center and promote the rapid progress of the reaction. The fluorine-containing additive can be tetrabutylammonium fluoride, cesium fluoride, potassium fluoride or a mixture thereof. An amount of the fluorine-containing additive can be a conventional amount for such reactions in the art. A molar ratio of the compound A-5 to the fluorine-containing additive can be 1:(0.1-1), preferably 1:(0.1-0.5).

The post-treatment of the method for synthesizing the compound A-6 can be a conventional post-treatment for such reactions in the art, and preferably comprises adding water and an ether solvent to the reaction solution after the completion of the coupling reaction at 0° C.-10° C., separating into layers at room temperature, washing the organic phase with saturated brine, drying (such as anhydrous sodium sulfate or anhydrous magnesium sulfate), filtering, and concentrating to obtain the compound A-6.

The method for synthesizing the compound A-6 can further comprise a method for synthesizing compound A-5, which preferably comprises conducting a formylation reaction as shown below with compound A-4 in the presence of DMF and POCl₃ to obtain the compound A-5;

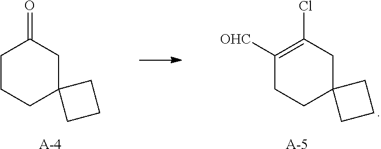

In the method for synthesizing the compound A-5, the conditions for the formylation reaction can be conventional conditions for such reactions in the art. In the present disclosure, the following conditions are preferred: a molar ratio of the compound A-4 to POCl₃ can be 1:(1-5), preferably 1:(1-3). A molar ratio of the compound A-4 to DMF can be 1:(1-5), preferably 1:(1-3). The solvent can be a chlorinated alkane solvent, such as dichloromethane, chloroform, 1,2-dichloroethane or a mixture thereof. A mass ratio of the compound A-4 to the solvent can be 1:(1-10), preferably 1:(5-10). The temperature of the formylation reaction can be the reflux temperature of the solvent under normal pressure. The progress of the formylation reaction can be detected by conventional methods in the art (e.g., TLC, GC, HPLC or NMR, etc.), and the disappearance of compound A-4 detected by TLC is generally regarded as the completion of the reaction. The time of the formylation reaction can be 1-5 hours, preferably 2-4 hours.

The method for synthesizing the compound A-5 preferably comprises adding POCl₃ to a mixture of DMF and a solvent (preferably under ice-water bath conditions), and stirring the mixture at room temperature (for example, stirring at 25° C.-35° C. for 1 hour), and then mixing with a mixed solution of the compound A-4 and the solvent (mixing temperature is preferably −5° C.-5° C.) to conduct the formylation reaction.

In the method for synthesizing the compound A-5, in the post-treatment operation, the pH value of the reaction solution needs to be controlled to about 5-6, which can effectively avoid a disproportionation reaction of the compound A-5.

The post-treatment in the method for synthesizing the compound A-5 preferably comprises adjusting the pH value of the reaction solution after the completion of the formylation reaction to 5-6 (preferably at −10 to 0° C., adjusting with a 20% NaOH aqueous solution, then stirring at room temperature for 20-30 minutes), separating into layers, extracting the aqueous phase with a chlorinated alkane solvent (such as dichloromethane), washing the organic phase with water, drying (such as anhydrous sodium sulfate or anhydrous magnesium sulfate), and concentrating to dryness to obtain the compound A-5.

The method for synthesizing the compound A-5 can further comprise a method for synthesizing compound A-4, which preferably comprises conducting a reduction reaction as shown below with compound A-3 in a solvent and in the presence of an organic acid, hydrogen and a metal catalyst to obtain the compound A-4;

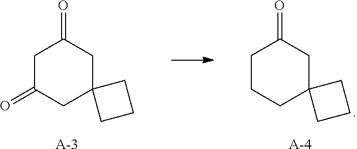

In the method for synthesizing the compound A-4, the organic acid can be a conventional organic acid for such reactions in the art, such as methanesulfonic acid, p-toluenesulfonic acid, acetic acid or a mixture thereof. An amount of the organic acid can be a conventional amount for such reactions in the art. A molar ratio of the compound A-3 to the organic acid can be 1:(0.01-0.5), preferably 1:(0.01-0.3).

In the method for synthesizing the compound A-4, the metal catalyst can be a conventional metal catalyst for such reactions in the art, for example, palladium, platinum, palladium on carbon, palladium acetate, palladium hydroxide or a mixture thereof (such as 10% Pd/C). An amount of the metal catalyst can be a conventional amount for such reactions in the art. A mass ratio of the compound A-3 to the metal catalyst can be 1:(0.01-0.1), preferably 1:(0.05-0.1).

In the method for synthesizing the compound A-4, the solvent can be a conventional solvent for such reactions in the art, for example, water, a C₁-C₆ alcohol solvent or a mixture thereof. The C₁-C₆ alcohol solvent can be methanol, ethanol, isopropanol, tert-butanol, n-butanol or a mixture thereof. An amount of the solvent may not be specifically limited as long as it does not affect the progress of the reaction, e.g., a mass ratio of the compound A-3 to the solvent can be 1:(1-10), preferably 1:(5-10).

In the method for synthesizing the compound A-4, the temperature of the reduction reaction can be a conventional temperature for such reactions in the art, and the reaction temperature can be 50-100° C., more preferably 50-85° C.

In the method for synthesizing the compound A-4, the pressure of hydrogen should be controlled within an appropriate range to prevent the side reaction caused by excessive hydrogenation. Generally, the pressure of hydrogen should be 0.5-0.6 MPa.

In the method for synthesizing the compound A-4, the progress of the reduction reaction can be detected by conventional methods in the art (e.g., TLC, GC, HPLC or NMR, etc.), and the disappearance of compound A-3 detected by TLC is generally regarded as the completion of the reaction. The time of the reduction reaction can be 1-7 hours, preferably 2-6 hours.

In the method for synthesizing the compound A-4, the reduction reaction is preferably conducted at 50-55° C. and a hydrogen pressure of 0.50-0.60 MPa for 1-2 hours, and then at 80-85° C. and a hydrogen pressure of 0.50-0.60 MPa for 3-4 hours.

The method for synthesizing the compound A-4 preferably comprises adding compound A-3, an organic acid, and a metal catalyst in a solvent successively, and then conducting the reduction reaction in a hydrogen atmosphere.

The post-treatment in the method for synthesizing the compound A-4 can be the conventional post-treatment for such reactions in the art, and preferably comprises filtering the reaction solution after the completion of the reduction reaction, concentrating the filtrate to dryness, and mixing the residue with a chlorinated alkane solvent and water, separating into layers, extracting the aqueous phase with a chlorinated alkane solvent, combining the organic phases, washing with water to a pH of 5-6, drying (for example, anhydrous sulfuric acid or anhydrous magnesium sulfate), filtering, the filtrate is fractionated under normal pressure, and the distillate at 45-50° C. is collected to obtain the compound A-4.

The method for synthesizing the compound A-4 can further comprise a method for synthesizing the compound A-3, which preferably comprises conducting a condensation reaction as shown below with compound A-2 and diethyl 1,3-acetonedicarboxylate in a solvent and in the presence of a base to obtain the compound A-3;

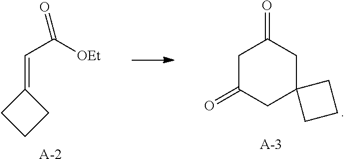

In the method for synthesizing the compound A-3, the conditions of the condensation reaction can be the conventional conditions for such reactions in the art. For example, the method disclosed in WO02016/37534.

The method for synthesizing the compound A-3 can further comprise a method for synthesizing the compound A-2, which preferably comprises conducting a Witting reaction as shown below with cyclobutanone and triethyl phosphonoacetate in a solvent and in the presence of a base to obtain the compound A-2;

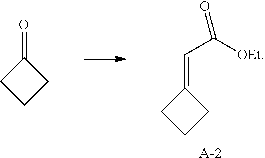

In the method for synthesizing the compound A-2, the conditions of the Witting reaction can be the conventional conditions for such reactions in the art. For example, the method disclosed in Journal of Journal of Organic Chemistry, 2016, Vol. 81 (3) p. 1057-1074.

The method for synthesizing the compound 1 can further comprise a method for synthesizing compound B, which preferably comprises conducting a nucleophilic substitution reaction as shown below with compound B-1 and 5-hydroxy-7-azaindole in a solvent and in the presence of a base to obtain the compound B;

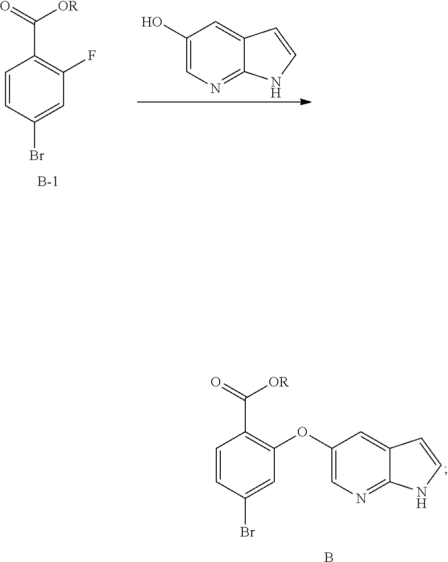

wherein R is C₁-C₈ alkyl.

In the method for synthesizing the compound B, the base can be a conventional base for such reactions in the art, for example, an inorganic base, an organic base or a mixture thereof. The inorganic base is, for example, potassium phosphate, sodium carbonate, sodium bicarbonate, potassium carbonate, sodium hydride or a mixture thereof. The organic base can be, for example, potassium tert-butoxide, sodium tert-butoxide or a mixture thereof. An amount of the base can be a conventional amount for such reactions in the art. A molar ratio of the compound B-1 to the base can be 1:(1-5), preferably 1:(1-2).

In the method for synthesizing the compound B, the solvent can be a conventional solvent for such reactions in the art, for example, a nitrile solvent, an amide solvent or a mixture thereof, preferably a mixed solvent of the nitrile solvent and the amide solvent. An amount of each solvent in the mixed solvent may not be specifically limited. Preferably, a volume ratio of the nitrile solvent to the amide solvent is 1:1-10:1, for example, 1:1-5:1. The nitrile solvent can be acetonitrile. The amide solvent can be N,N-dimethylformamide (DMF). The amount of the solvent may not be specifically limited as long as it does not affect the progress of the reaction. For example, a volume-mass ratio of the solvent to the compound B-1 can be 2 mL/g-20 mL/g, preferably 10 mL/g-20 mL/g.

In the method for synthesizing the compound B, a molar ratio of the compound B-1 to 5-hydroxy-7-azaindole can be 1:(1-5), preferably 1:(1-2).

In the method for synthesizing the compound B, the temperature of the nucleophilic substitution reaction can be 50-100° C., preferably 70-100° C.

In the method for synthesizing the compound B, the progress of the nucleophilic substitution reaction can be detected by conventional methods in the art (e.g., TLC, GC, HPLC or NMR, etc.), and the disappearance of compound B-1 detected by HPLC is generally regarded as the completion of the reaction. The time of the nucleophilic substitution reaction can be 1-24 hours, preferably 10-24 hours.

The post-treatment in the method for synthesizing the compound B can be conventional post-treatment for such reactions in the art, and preferably comprises mixing the reaction solution after the completion of the nucleophilic substitution reaction with water (such as mixing at room temperature, preferably stirring for 3-10 hours after mixing, the appropriate amount of water is no more solid precipitation), filtering (such as suction), washing the filter cake with water, mixing the filter cake with an ester solvent (such as ethyl acetate, the appropriate amount of the ester solvent is to dissolve the filter cake), washing with saturated brine, separating into layers, and concentrating the organic phase to dryness to obtain a crude product; recrystallizing (a solvent for recrystallization such as an ester solvent, an alkane solvents or a mixture thereof, such as ethyl acetate, n-heptane or a mixture thereof), filtering and drying to obtain the compound B.

The method for synthesizing the compound B can further comprise a method for synthesizing a compound B-1, which preferably comprises conducting an esterification reaction as shown below with 2-fluoro-4-bromobenzoic acid and alcohol ROH in a solvent and in the presence of a condensing agent to obtain the compound B-1;

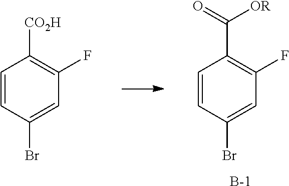

wherein R is C₁-C₈ alkyl.

In the method for synthesizing the compound B-1, the condensing agent can be a conventional condensing agent for such reactions in the art, for example, EDC.HCl (1-ethyl-3-(3-dimethylaminopropyl)carbodiimide hydrochloride), CDI (N,N′-carbonyl diimidazole), DCC (dicyclohexylcarbodiimide), HOBt (1-hydroxybenzotriazole), HOAT (1-hydroxy-7-aza-benzotriazole) or a mixture thereof, preferably a mixture of two or more condensing agents, such as a mixture of EDC.HCl and HOBt. When the condensing agent is a mixture of two or more kinds, an amount of each condensing agent in the mixture may not be specifically limited. Alternatively, a mass ratio of EDC.HCl to HOBt can be 1:1-5:1, such as 1: 1-2:1.

In the method for synthesizing the compound B-1, a molar ratio of 2-fluoro-4-bromobenzoic acid to the condensing agent can be 1:(1-5), preferably 1:(1-4).

In the method for synthesizing the compound B-1, the solvent can be a conventional solvent for such reactions in the art, for example, a chlorinated alkane solvent, such as dichloromethane, chloroform, 1,2-dichloroethane or a mixture thereof. An amount of the solvent may not be specifically limited as long as it does not affect the progress of the reaction. A mass-volume ratio of 2-fluoro-4-bromobenzoic acid to the solvent can be 1 mL/g-10 mL/g, preferably 5 mL/g-10 mL/g.

In the method for synthesizing the compound B-1, a molar ratio of 2-fluoro-4-bromobenzoic acid to the alcohol can be 1:(1-10), preferably 1:(1-8).

In the method for synthesizing the compound B-1, the temperature of the esterification reaction can be a conventional temperature for such reactions in the art, preferably room temperature to 50° C.

In the method for synthesizing the compound B-1, the progress of the esterification reaction can be detected by conventional methods in the art (e.g., TLC, GC, HPLC or NMR, etc.), and the disappearance of 2-fluoro-4-bromobenzoic acid detected by HPLC is generally regarded as the completion of the reaction. The time of the esterification reaction can be 1-24 hours, more preferably 10-24 hours.

In the second aspect, the present disclosure also provides a method for synthesizing compound 2, which is method 1 or method 2 as shown below.

The method 1 preferably comprises conducting a hydrolysis reaction as shown below with compound 1 in a solvent and in the presence of an acid or a base to obtain the compound 2;

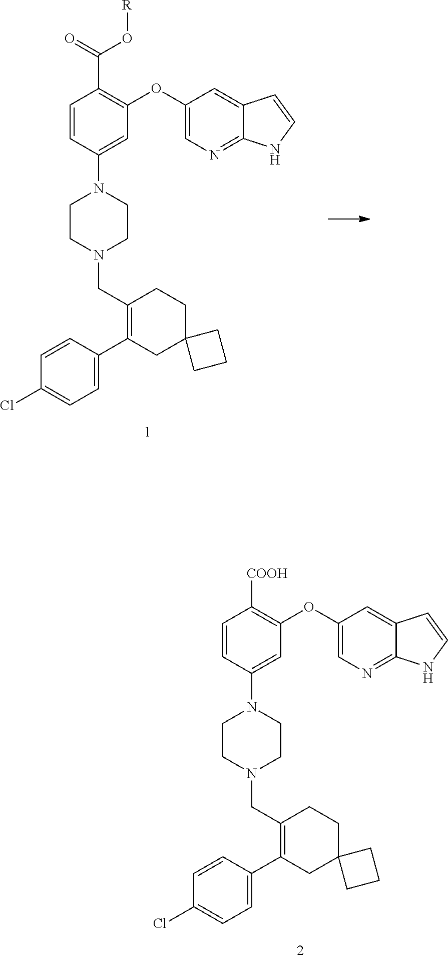

wherein, R is C₁-C₈ alkyl.

In the method 1, the acid can be a conventional acid for such reactions in the art, for example, an inorganic acid, an organic acid or a mixture thereof. The inorganic acid can be hydrochloric acid, sulfuric acid or a mixture thereof. The organic acid can be acetic acid, trifluoroacetic acid or a mixture thereof. A molar ratio of the acid to the compound A can be 1:(0.5-1), preferably 1:(0.5-0.8).

In the method 1, the base can be a conventional base for such reactions in the art, such as an inorganic base, an organic base or a mixture thereof. The inorganic base can be an alkali metal hydroxide, an alkali metal carbonate, an alkali metal bicarbonate or a mixture thereof, and also can be sodium hydroxide, potassium hydroxide, sodium carbonate, sodium bicarbonate, lithium carbonate, cesium carbonate or a mixture thereof. The organic base can be triethylamine, pyridine, DBU, DIPEA, triethylenediamine (DABCO), DBN, DMAP, N-methylmorpholine, tetramethylethylenediamine, potassium tert-butoxide, tert-butanol sodium, n-butyllithium, KHMDS, NaHMDS, LDA, potassium tert-pentoxide, sodium tert-pentoxide or a mixture thereof. A molar ratio of the base to the compound A can be 1:(0.5-1), preferably 1:(0.5-0.8).

In the method 1, the solvent can be a conventional solvent for such reactions in the art, such as water, an ether solvent or a mixture thereof. The ether solvent can be diethyl ether, tetrahydrofuran, 2-methyltetrahydrofuran, 1,4-dioxane, methyl tert-butyl ether or a mixture thereof. An amount of the solvent may not be specifically limited as long as it does not affect the progress of the reaction. A mass ratio of the compound 1 to the solvent can be 1:(1-10), preferably 1:(5-10).

In the method 1, the temperature of the hydrolysis reaction can be a conventional temperature for such reactions in the art, for example, room temperature to 60° C., preferably 50-60° C.

In the method 1, the progress of the hydrolysis reaction can be detected by conventional methods in the art (e.g., TLC, GC, HPLC or NMR, etc.), and the disappearance of compound 1 detected by HPLC is generally regarded as the completion of the reaction. The time of the hydrolysis reaction can be 1-5 hours, preferably 2-4 hours.

In the method 1, the method for synthesizing the compound 1 is preferably as described above.

The method 2 preferably comprises conducting a Buchwald-Hartwig coupling reaction as shown below with a compound C and the compound A in a solvent and in the presence of a base and a palladium catalyst to obtain the compound 2;

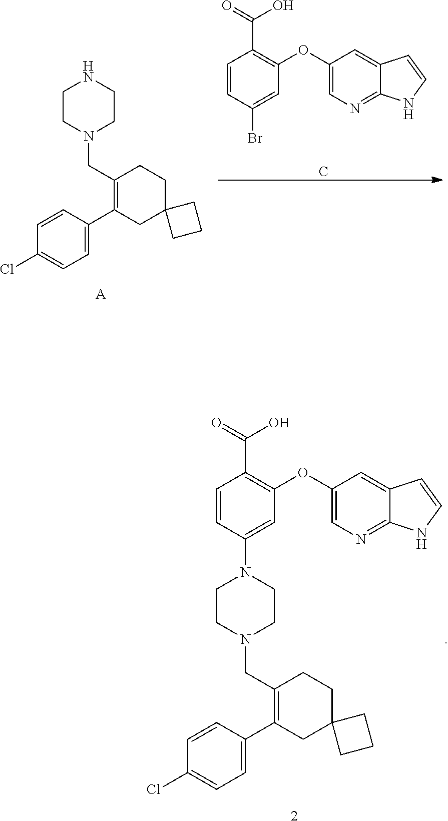

In the method 2, the conditions of the Buchwald-Hartwig coupling reaction are the same as the conditions of the Buchwald-Hartwig coupling reaction in the method for synthesizing the compound 1.

In the third aspect, the present disclosure also provides a method for synthesizing the compound 3, which comprises conducting an amidation reaction as shown below with compound 2 and compound D in the presence of a condensing agent, a base and a catalyst to obtain the compound 3;

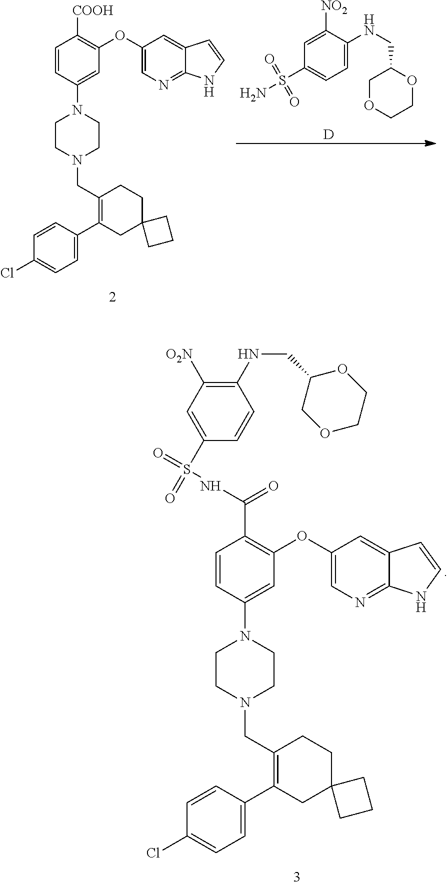

In the method for synthesizing the compound 3, the condensing agent can be a conventional condensing agent for such reactions in the art, such as DCC (dicyclohexylcarbodiimide), EDC.HCl (1-ethyl-3-(3-dimethylaminopropyl)carbodiimide hydrochloride), CDI (N,N′-carbonyl diimidazole), HATU (2-(7-azabenzotriazol-1-yl)-N,N,N′,N′-tetramethyluronium hexafluorophosphate), HBTU (benzotriazole-N,N,N′,N′-tetramethyluronium hexafluorophosphate), TBTU (O-benzotriazole-N,N,N′,N′-tetramethyluronium tetrafluoroborate), HOBT (1-hydroxybenzotriazole), HOAT (1-hydroxy-7-aza-benzotriazole) or a mixture thereof. A molar ratio of the compound 2 to the condensing agent can be 1:(1-2), preferably 1:(1-1.5).

In the method for synthesizing the compound 3, the base can be a conventional base for such reactions in the art, such as an inorganic base, an organic base or a mixture thereof. The inorganic base can be an alkali metal hydroxide, an alkali metal carbonate, an alkali metal bicarbonate or a mixture thereof, and also can be sodium hydroxide, potassium hydroxide, sodium carbonate, sodium bicarbonate, lithium carbonate, cesium carbonate or a mixture thereof. The organic base can be triethylamine, N,N-diisopropylethylamine, pyridine or a mixture thereof. A molar ratio of the compound 2 to the base can be 1:(1-5), preferably 1:(1-2).

In the method for synthesizing the compound 3, the catalyst can be a conventional catalyst for such reactions in the art, such as DMAP (4-dimethylaminopyridine). A mass ratio of the compound 2 to the catalyst can be 1:(0.1-1), preferably 1:(0.1-0.5).

In the method for synthesizing the compound 3, a molar ratio of the compound 2 to the compound D can be 1:(0.8-1.5), preferably 1:(0.8-1.1).

In the method for synthesizing the compound 3, the temperature of the amidation reaction can be a conventional temperature for such reaction in the art, for example: 20-50° C., preferably room temperature.

In the method for synthesizing the compound 3, the progress of the amidation reaction can be detected by conventional methods in the art (e.g., TLC, GC, HPLC or NMR, etc.), and the disappearance of compound 2 detected by HPLC is generally regarded as the completion of the reaction. The time of the amidation reaction is 1-24 hours, more preferably 5-24 hours.

In the method for synthesizing the compound 3, after the amidation reaction is completed, conventional post-treatment methods for such reactions in the art can be used for the post-treatment, such as recrystallization. That is, a crude product of the compound 3 can be purified by recrystallization.

A solvent for recrystallization can be a chlorinated alkane solvent, an ether solvent or a mixture thereof, preferably a mixed solvent of the chlorinated alkane solvent and the ether solvent. An amount of each solvent in the mixed solvent may not be specifically limited. Preferably, a volume ratio of the chlorinated alkane solvent to the ether solvent is 1:1-1:10, for example, 1:1-1:5. The chlorinated alkane solvent can be dichloromethane, chloroform, 1,2-dichloroethane or a mixture thereof. The ether solvent can be tetrahydrofuran, 2-methyltetrahydrofuran, 1,4-dioxane, methyl tert-butyl ether or a mixture thereof. A volume-mass ratio of the solvent used for recrystallization to the crude product of the compound 3 can be 10 mL/g-100 mL/g, preferably 30 mL/g-50 mL/g. The temperature of the recrystallization can be room temperature. The time of the recrystallization may not be specifically limited, for example, it can be 0.5-5 hours, preferably 1-3 hours.

Therefore, in one aspect, the present disclosure also provides a method for purifying the compound 3, which comprises recrystallizing the crude product of the compound 3. The conditions of the recrystallization are as described above. The HPLC purity of the crude product of the compound 3 is preferably <99%, .e.g., not less than 90%. The crude product of the compound 3 can be prepared by conventional methods in the art, and is preferably prepared by the method of the present disclosure. In the present disclosure, the yield of the purified product of the compound 3 obtained by the purification method of the compound 3 is 70%-85%, and the HPLC purity is above 99%. In the method for synthesizing the compound 3, the method for synthesizing the compound 2 can be as described above.

The method for synthesizing the compound 3 can further comprise a method for synthesizing the compound D, which preferably comprises conducting a reaction as shown below with a compound D-1 and (S)-2-aminomethyl-1,4-dioxane hydrochloride in a solvent and in the presence of a base to obtain the compound D;

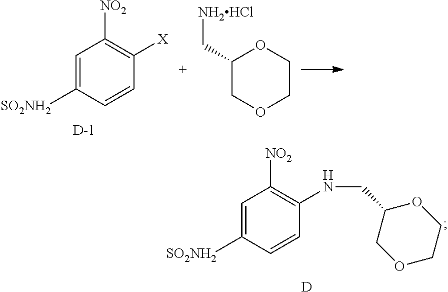

wherein X is halogen, preferably F or Cl.

In the method for synthesizing the compound D, the solvent can be selected from a nitrile solvent. The nitrile solvent can be acetonitrile. A volume-mass ratio of the solvent to the compound D-1 is 10 mL/g-20 mL/g, preferably 15 mL/g-20 mL/g.

In the method for synthesizing the compound D, the base can be a conventional base for such reactions in the art, for example, an inorganic base, an organic base or a mixture thereof. The inorganic base can be an alkali metal hydroxide, an alkali metal carbonate or a mixture thereof, as also can be sodium hydroxide, potassium hydroxide, potassium carbonate, sodium carbonate or a mixture thereof. The organic base can be, for example, triethylamine, pyridine, DBU, DIPEA, triethylenediamine (DABCO), DBN, DMAP, N,N-diisopropylethylamine, N-methylmorpholine, tetramethyl ethylenediamine, potassium tert-butoxide, sodium tert-butoxide, n-butyl lithium, KHMDS, NaHMDS, LDA or a mixture thereof. A molar ratio of the compound D-1 to the base can be 1:(1-10), preferably 1:(1-5).

In the method for synthesizing the compound D, a molar ratio of the compound D-1 to (S)-2-aminomethyl-1,4-dioxane hydrochloride can be 1:(1-2), preferably 1:(1-1.2).

In the method for synthesizing the compound D, the reaction temperature can be a conventional temperature for such reactions in the art, for example, room temperature to the solvent reflux temperature under normal pressure.

In the method for synthesizing the compound D, the progress of the reaction can be detected by conventional methods in the art (e.g., TLC, GC, HPLC or NMR, etc.). The disappearance of compound 1 detected by HPLC or TLC is generally regarded as the completion of the reaction. The reaction time can be 1-72 hours, more preferably 24-72 hours.

In the fourth aspect, the present disclosure also provides a method for synthesizing the salt of the compound A, which comprises conducting a reaction as shown below with compound A-7 in a solvent and in the presence of an acid to obtain the salt of the compound A;

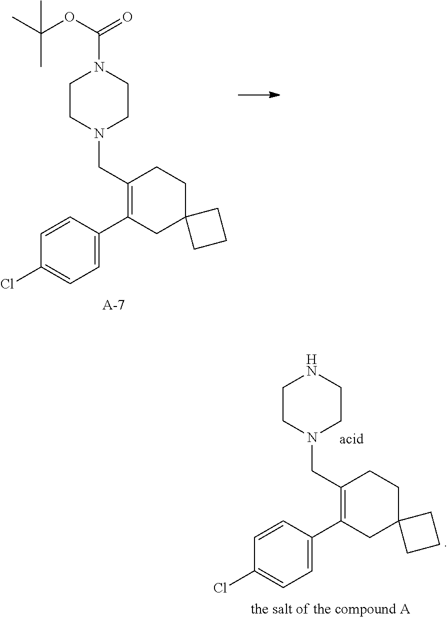

The conditions of the method for synthesizing the salt of the compound Aare the same as those described above.

In the fifth aspect, the present disclosure also provides a method for synthesizing the compound B, which comprises conducting a nucleophilic substitution reaction as shown below with compound B-1 and 5-hydroxy-7-azaindole in the presence of a solvent and a base to obtain the compound B;

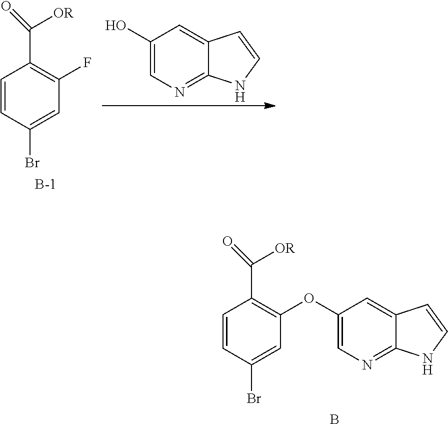

wherein R is C₁-C₈ alkyl.

The conditions of the method for synthesizing the compound B are the same as those described above.

In the sixth aspect, the present disclosure also provides a method for synthesizing the compound D, which comprises conducting a reaction as shown below with the compound D-1 and (S)-2-aminomethyl-1,4-dioxane hydrochloride in a solvent and in the presence of a base to obtain the compound D,

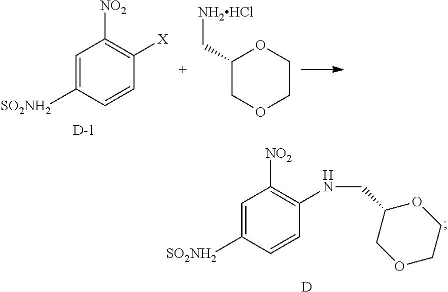

wherein X is halogen, preferably F or Cl.

The conditions of the method for synthesizing the compound D are the same as those described above.

In the present disclosure, the synthetic route of the compound 3 is as follows:

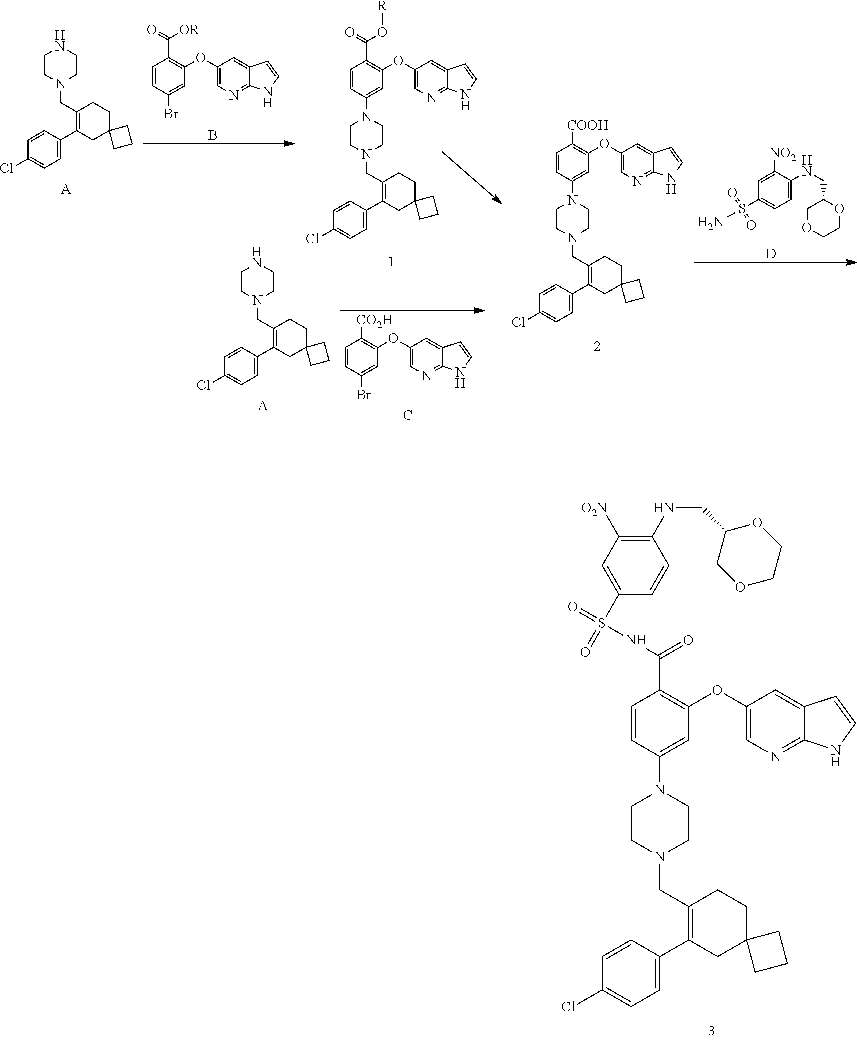

the conditions of each step of the reaction are the same as those described above.

In the present disclosure, the synthetic route of the compound A is as follows:

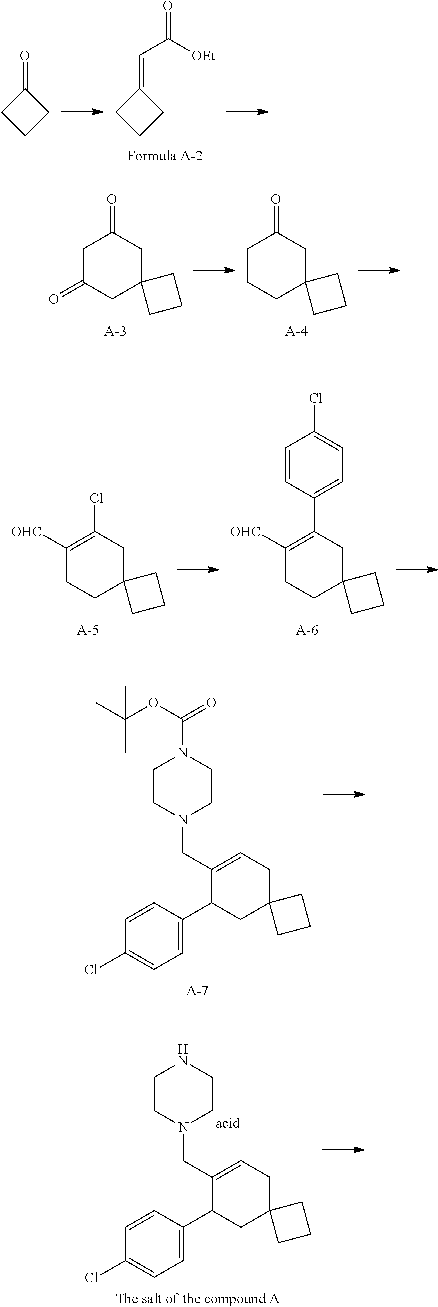

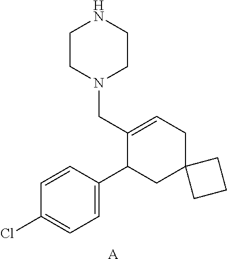

the conditions of each step of the reaction are the same as those described above.

In the present disclosure, the synthetic route of the compound B is as follows:

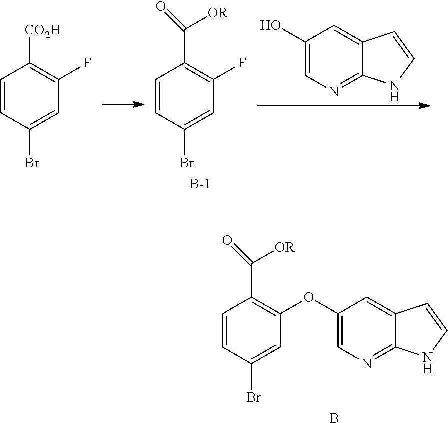

the conditions of each step of the reaction are the same as those described above.

The present disclosure also provides compounds as shown below or pharmaceutically acceptable salts thereof:

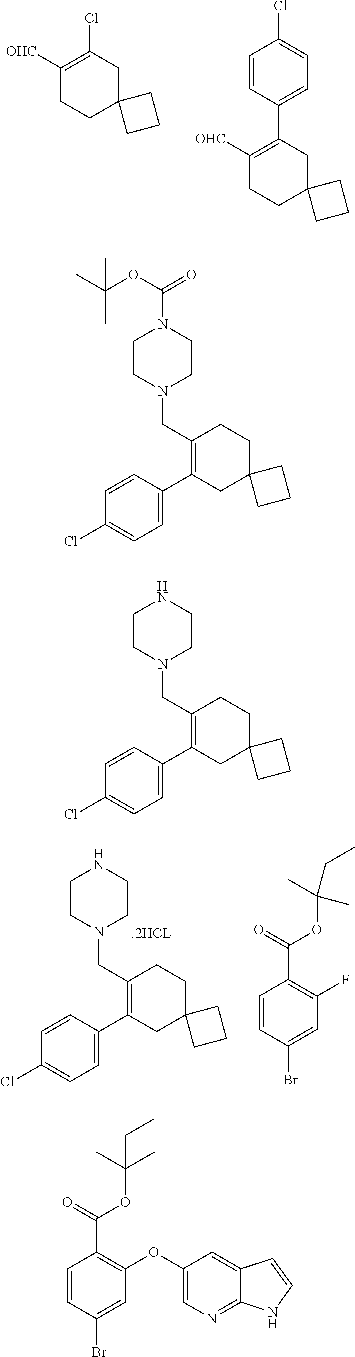

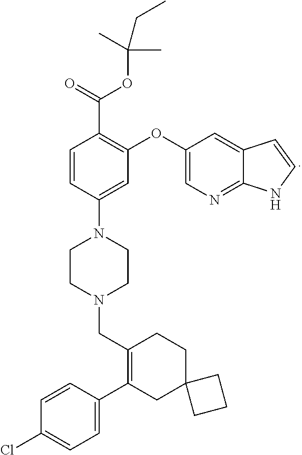 [SMILES: CCC(C)(C)OC(=O)c1ccc(N2CCN(CC3=C(c4ccc(Cl)cc4)CC4(CCC4)CC3)CC2)cc1Oc1cnc2[nH]ccc2c1]

In the present disclosure, the term C₁-C₈ alkyl refers to linear or branched C₁-C₈ alkyl, preferably linear or branched C₁-C₅ alkyl. For example, methyl, ethyl, n-propyl, isopropyl, n-butyl, isobutyl, tert-butyl, sec-butyl, n-pentyl, isopentyl or neopentyl.

In the present disclosure, the term halogen refers to F, Cl, Br or I.

In the present disclosure, the room temperature refers to 0-40° C., preferably 10-30° C., more preferably 25° C.

In the present disclosure, the normal pressure refers to one standard atmospheric pressure.

Without violating the common knowledge in the art, the preferred parameters mentioned above may be optionally combined to obtain the preferred embodiments in the present disclosure.

The reagents and raw materials employed in the present disclosure are commercially available.

The advantageous effects achieved by the present disclosure are as follows:

1. The present disclosure synthesizes three intermediate compounds required by the target compound and their preparation methods for the first time. The above route involves many new intermediate compounds synthesized for the first time.

2. The target compound 3 is synthesized by using the three intermediates synthesized by the present disclosure, which has the advantages of low-cost, easy-to-obtain reaction raw materials, mild and controllable reaction conditions, green synthesis process, and suitable for industrial production.

### DETAILED DESCRIPTION OF THE PREFERRED EMBODIMENT

The following examples further illustrate the present disclosure, but the present disclosure is not limited thereto. In the following examples, the experimental methods without specific conditions are selected according to conventional methods and conditions, or according to product specifications.

In the following examples, if the temperature is not specified, it means that it is conducted at room temperature.

### Example 1 Synthesis of Hydrochloride Salt of the Compound A

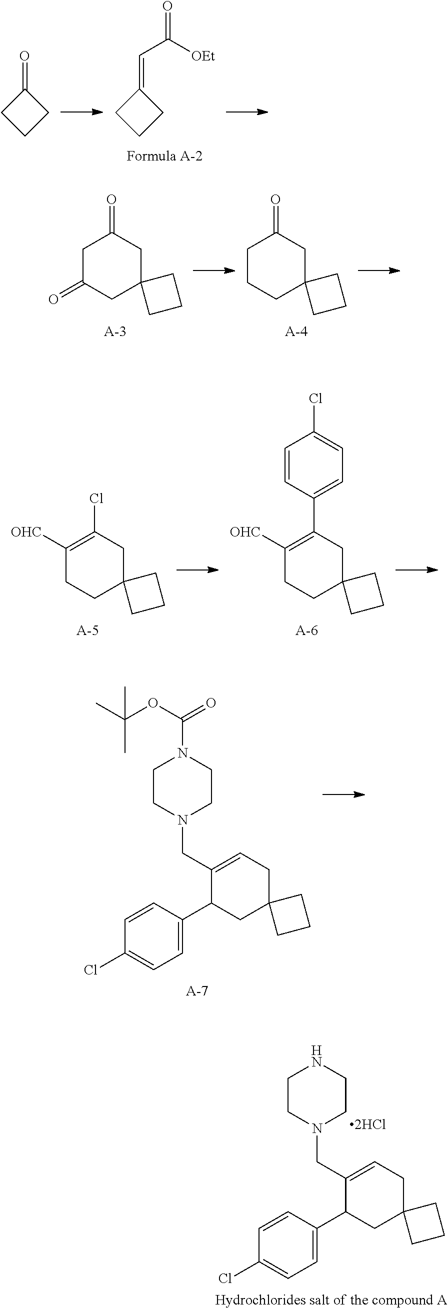

Step 1 Synthesis of the Compound A-2

At room temperature, methyl tert-butyl ether (126 mL) was added to a reactor, cyclobutanone (18 g) and triethyl phosphonoacetate (57 g) were added successively under stirring, the reaction temperature was raised to about 50° C.-60° C., potassium hydroxide (17 g) was added. After the addition was completed, the reaction mixture was cooled to room temperature and stirred for 10-12 hours, a sample was taken and in-process control of the reaction was performed. After the reaction was completed, the reaction was quenched with dilute hydrochloric acid. After separation, the aqueous phase was extracted with methyl tert-butyl ether, the organic phases were combined and washed with saturated brine, dried over anhydrous sodium sulfate, concentrated, and 35 g of the compound A-2 was obtained as a yellow oil.

Step 2 Synthesis of the Compound A-3

At room temperature, THF (400 mL) was added to a reactor, sodium hydride (24 g) was added at first, and then diethyl acetonedicarboxylate (61 g) was added, the mixture was stirred, after that the compound A-2 (35 g) and anhydrous ethanol (12 g) were added. Then the reaction solution was heated to 60° C.-70° C., stirred for 1-2 hours, then cooled to room temperature, added with methanol and 20% potassium hydroxide aqueous solution (280 g) successively. And the mixture was heated to 60° C.-70° C. and continued to stir for 12 hours, then cooled to room temperature, added with methyl tert-butyl ether for extraction, the organic phase was discarded to obtain an aqueous phase, which was acidified with concentrated hydrochloric acid at 40° C. Then the reaction solution was heated to 45° C.-50° C. and stirred for 3 hours. After the reaction was completed, the reaction solution was cooled to 0° C.-10° C., filtered and concentrated to obtain a crude product by slurrying with demineralized water, concentrated, dried under vacuum to obtain the compound 3 as an off-white solid (18 g, yield 53%).

¹HNMR (300 MHz, CDCl₃): δ ppm 2.94 (2H, d, J=1.6), 2.52 (2H, s), 1.79-2.03 (6H, m).

Step 3 Synthesis of the Compound A-4

Methanol (121 g) was added to a hydrogenation reactor, compound A-3 (20 g, 0.132 mol) was added, and the mixture was dissolved by stirring. Then acetic acid (0.845 g, 0.014 mol) and 10% Pd/C (1.41 g) were added successively. The mixture was replaced with nitrogen, then replaced with hydrogen, and a hydrogenation reduction was conducted for 3-4 hours, a sample was taken and in-process control of the reaction was performed until the reaction was completed. After filtration, the filtrate was concentrated to dryness to obtain a residue. Then dichloromethane and water were added and separated to obtain an organic phase, then dichloromethane was added to extract the aqueous phase. The organic phases were combined, washed with water until the pH of the aqueous phase was 5-6, dried over anhydrous sodium sulfate, filtered, and the filtrate was fractionated at atmospheric pressure, the distillate at 45-50° C. was collected to obtain 13 g of colorless transparent liquid, with a GC purity of 89%, and a yield of 73.4%.

¹HNMR (400 MHz, CDCl₃): δ ppm 2.38 (2H, s), 2.19-2.22 (2H, m), 1.75-1.89 (10H, m).

Step 4 Synthesis of the Compound A-5

DMF (29 g, 0.40 mol) and CH₂Cl₂ (161 g) were added to a reactor, POCl₃ (54 g, 0.35 mol) was added dropwise at −5-5° C. in an ice-water bath with stirring. After the addition was completed, the reaction was heated to 25° C.-35° C. and stirred at 25° C.-35° C. for 1 hour, and then cooled to −5° C.-5° C., and a solution of the compound A-4 (20 g, 0.15 mol) in CH₂Cl₂ (20 g) was slowly added dropwise. After the addition, the reaction was heated and refluxed for 2-4 hours, then cooled to −10 to 0° C., added dropwise with 20% NaOH aqueous solution, adjusted the pH of the aqueous phase to 5-6, raised to room temperature and stirred for 20-30 minutes, separated. The aqueous phase was extracted with dichloromethane. The organic phase was washed with water and dried over anhydrous sodium sulfate, the filtrate was concentrated to dryness to obtain 24.6 g of oil, and the oil was directly conducted the next reaction without purification.

Step 5 Synthesis of the Compound A-6

The compound A-5 (25 g, 0.135 mol), ethylene glycol dimethyl ether (131 g), water (61 g) and ethanol (35 g) were added to a reactor successively. The mixture was stirred for 20-30 minutes, added with potassium carbonate (54 g, 0.39 mol), replaced with nitrogen, added with Pd(PPh₃)₂Cl₂ (0.9 g, 0.0013 mol) under the protection of nitrogen, heated to 40° C.-50° C., added dropwise with a solution of p-chlorophenylboronic acid (p-chlorophenylboronic acid (16 g, 0.103 mol)+DME (26 g)+EtOH (6 g)+H₂O (12 g)). After the addition, the reaction temperature was maintained for 1-2 hours. After the reaction was completed, the reaction solution was cooled to 0° C.-10° C., added with water and methyl tert-butyl ether, and separated at room temperature. The organic phase was washed with saturated brine, dried over anhydrous sodium sulfate, filtered, and concentrated under reduced pressure to obtain 22 g of oil.

Step 6 Synthesis of the Compound A-7

1-Boc-piperazine (32 g, 0.13 mol) was added to a reactor, replaced with nitrogen, then the compound A-6 (22 g, 0.085 mol) and dichloromethane (200 g) were added, and the reaction solution was stirred at 25-35° C. for 40-60 minutes, NaBH(OAc)₃ (84 g, 0.40 mol) was added in batches at 25-35° C. After the addition, the reaction was continued for 2-3 hours, then cooled to −5-5° C., added with water and 20% NaOH aqueous solution slowly, adjusted the pH to 7-8, and separated. The organic phase was added with activated carbon and stirred under reflux for 0.5-1.5 hours, filtered while hot, and the filtrate was concentrated to dryness under reduced pressure. The crude product is recrystallized with acetonitrile and filtered to obtain a solid and dried under vacuum.

¹H NMR (400 MHz, DMSO-d₆) δ ppm: 7.38 (2H, d, J=8.4), 7.09 (2H, d, J=8.4), 3.23 (4H, m), 2.69 (2H, s), 2.16 (2H, m), 2.03 (2H, m), 1.73-1.88 (6H, m), 1.63 (2H, t, J₁=12.4, J₂=6.4), 1.35 (9H, s).

A total yield of the compound A-4 to the compound A-7 was about 79%.

Step 7 Synthesis of the Hydrochloride Salt of the Compound A

The compound A-7 (33 g) and isopropanol (260 g) were added to a reactor, the reaction temperature was controlled at 0-30° C., then concentrated hydrochloric acid (28 g) was added dropwise. After the addition, the reaction solution was raised to 60-70° C., stirred, a sample was taken and in-process control of the reaction was performed until the reaction was completed. The reaction solution was cooled to 0-10° C., filtered to obtain 36 g of a crude product, the crude product was recrystallized with a mixed solvent of isopropanol (180 g) and water (13 g), filtered, and the solid was dried under vacuum at 45-50° C. to obtain the hydrochloride salt of the compound A (31 g).

¹HNMR: (400 MHz, DMSO): δ ppm 11.62 (1H, s), 9.79 (1H, s), 9.46 (1H, s), 9.46 (2H, d, J=8.4), 7.18 (2H, d, J=8.0), 3.38-3.53 (8H, m), 2.88 (2H, s), 2.42 (2H, m), 2.33 (2H, m), 1.73-1.93 (6H, m), 1.69 (2H, t, J₁=12.4, J₂=6.4).

### Example 2 Synthesis of the Compound B

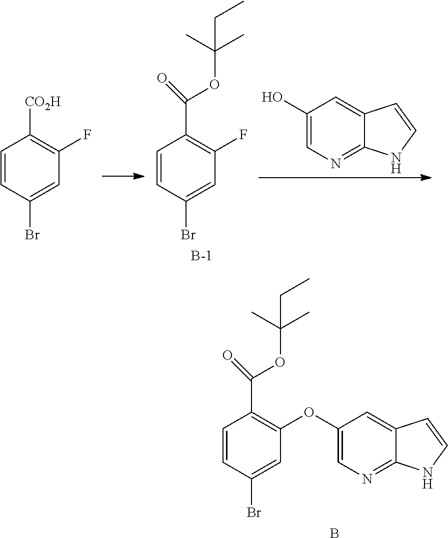

Step 1 Synthesis of the Compound B-1 (Tert-Amyl 2-Fluoro-4-Bromobenzoate)

2-Fluoro-4-bromobenzoic acid (30 g, 0.138 mol), dichloromethane (240 mL), EDC.HCl (52 g, 0.271 mol), HOBt (33 g, 0.244 mol) and tert-amyl alcohol (72 g, 0.818 mol) were added to a three-neck flask, the mixture was stirred at room temperature, a sample was taken and in-process control of the reaction was performed until the reaction was completed. The reaction mixture was adjusted to pH 1-2 with dilute hydrochloric acid, separated, the organic phase was washed with saturated NaHCO₃ solution, saturated brine successively, and dried over anhydrous sodium sulfate, filtered and concentrated to dryness to obtain 33 g of oil, with a yield of 84% and a purity of 97%.

¹HNMR (400 MHz, CDCl₃): δ ppm 7.75 (1H, t, J=8.0), 7.68 (1H, d, J=10.4), 7.54 (1H, dd, J₁=8.4, J₂=2.0), 1.83 (2H, q, J₁=22.4, J₂=15.2, J₃=7.6), 1.39 (6H, s), 0.90 (1H, t, J=7.2).

Step 2 Synthesis of the Compound B

5-Hydroxy-7-azaindole (5 g, 0.037 mol), the compound B-1 (10 g, 0.035 mol), a mixed solvent of DMF and acetonitrile (70 mL/70 mL) and potassium phosphate (10 g) were added to a three-neck flask, the reaction temperature was controlled at 90-95° C. under the protection of N₂, a sample was taken and in-process control of the reaction was performed until the reaction was completed. The reaction solution was cooled to 10° C., water (400 mL) was added, a solid was precipitated. The mixture was stirred for 5 hours, filtered with suction, and the filter cake was washed with water. In addition, ethyl acetate (300 mL) was added to dissolve the filter cake, then the solution was washed with saturated brine, separated, and the organic phase was concentrated to dryness to obtain 12.89 g of a crude product. The crude product was recrystallized with ethyl acetate and n-heptane and filtered. The filter cake was dried at 40° C. to obtain the compound B (10.94 g) with a yield of 78%.

¹HNMR (400 MHz, CDCl₃): δ ppm 11.76 (1H, s), 8.05 (1H, d, J=2.4), 7.68 (1H, d, J=8.4), 7.62 (1H, d, J=2.8), 7.54 (1H, t, J₁=6, J₂=2.8), 7.41 (1H, dd, J₁=8.4, J₂=1.6), 7.04 (1H, d, J=2.0), 6.44 (1H, q, J₁=4.8, J₂=3.2, J₃=2), 1.75 (2H, q, J₁=22.4, J₂=14.8, J₃=7.6), 1.39 (6H, s), 0.82 (1H, t, J=7.2).

### Example 3 Synthesis of the Compound D

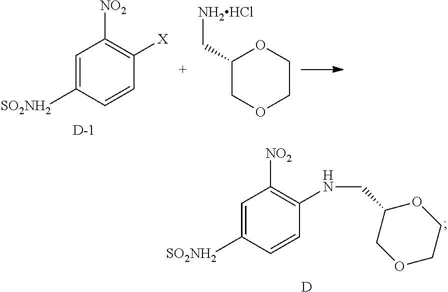

Acetonitrile (1.2 L), (S)-2-aminomethyl-1,4-dioxane hydrochloride (60 g, 0.39 mol), 4-chloro-3-nitrobenzene sulfonamide (92 g, 0.39 mol) and N, N-diisopropylethylenediamine (152 g, 1.17 molmol) were added to a reactor successively, the reaction solution was heated to the reflux temperature of acetonitrile, a sample was taken and in-process control of the reaction was performed until the reaction was completed. The reaction solution was cooled to room temperature, stirred for 30 minutes, filtered, and the filter cake was dried under vacuum at 50° C. to obtain the compound D with a yield of 78% and a purity of not less than 99%.

¹HNMR (400 MHz, CDCl₃): δ ppm 8.53 (1H, t, J=5.2), 8.48 (1H, d, J=2.0), 7.84 (1H, dd, J₁=9.2, J₂=2), 7.35 (2H, s), 7.27 (1H, d, J=8.8), 3.77-3.81 (3H, m), 3.40-3.67 (5H, m), 3.32-3.35 (1H, m).

### Example 4 Synthesis of the Compound 3

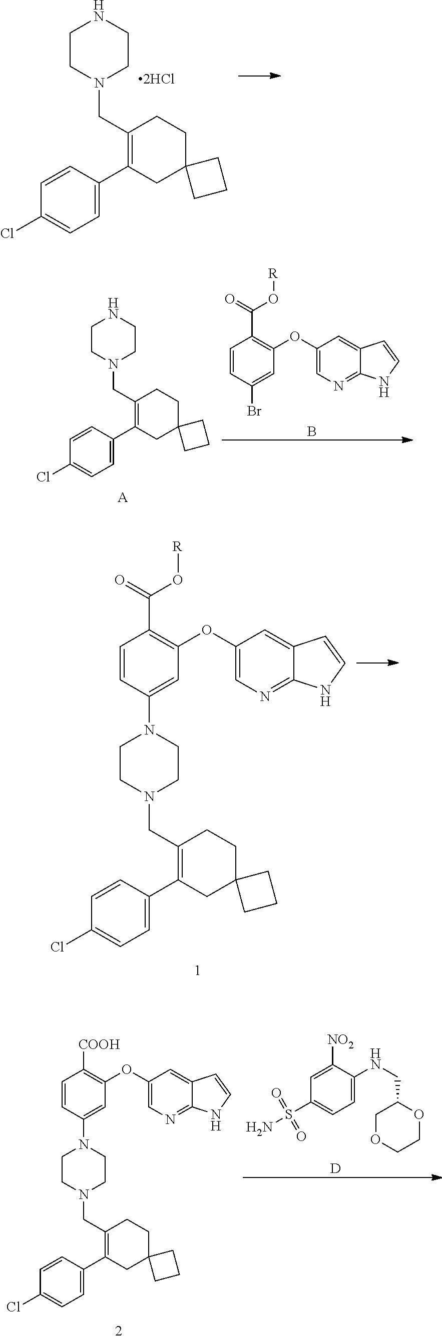

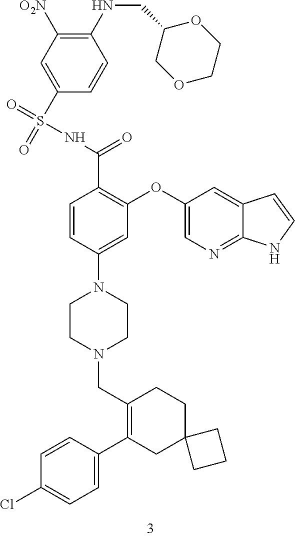 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

Step 1 Synthesis of the Compound 1

The hydrochloride salt of the compound A (2.5 g) and water (12 g) were added to a reactor. The mixture was stirred to dissolve, added with toluene (14 g), added with saturated sodium bicarbonate aqueous solution, adjusted the pH of the aqueous phase to 8-9, separated to obtain a toluene phase, the aqueous phase was extracted with toluene (14 g). The toluene phases were combined and washed with saturated brine, dried over anhydrous sodium sulfate, concentrated under reduced pressure, and water was removed with toluene to obtain the compound A (2 g).

Toluene (10.30 g), tetrahydrofuran (5.90 g), the compound B (2.87 g) were added to a reactor, replaced with nitrogen, bis(di-tert-butyl(4-(dimethylaminophenyl)phosphine)dichloropalladium(II) (0.54 g, 7 mmol) and sodium tert-pentoxide (2.91 g) were added under the protection of nitrogen. The reaction mixture was heated and stirred for about 1.0 hour, and then cooled to room temperature. The reaction solution was adjusted to pH with dilute hydrochloric acid, extracted with ethyl acetate, and the organic phase was stirred with N-acetyl-L-cysteine sodium bicarbonate aqueous solution (N-acetyl-L-cysteine:sodium bicarbonate:water=1:1.1:12.8 mass ratio) three times, separated, the organic phase was washed with saturated brine, dried over anhydrous sodium sulfate, and concentrated to obtain 3.4 g of the compound 1 with a yield of 70%.

¹HNMR (400 MHz, d-DMSO): δ ppm 11.58 (1H, s), 7.96 (1H, d, J=2.4), 7.66 (1H, d, J=8.8), 7.46 (1H, m), 7.35 (3H, m), 7.08 (2H, d, J=8.4), 6.75 (1H, dd, J₁=9.2, J₂=1.2), 6.36 (2H, m), 3.11 (4H, m), 2.71 (2H, m), 2.26 (2H, m), 2.18 (6H, m), 1.62-1.88 (8H, m), 1.58 (2H, m), 1.28 (6H, s), 0.71-0.75 (3H, t, J=7.6).

Step 2 Synthesis of the Compound 2

A solution of the compound 1 (20 g) dissolved in tetrahydrofuran (140 g) was added to a reactor, then potassium tert-butoxide (16 g) and water (16 g) were added slowly, the reaction solution was heated to 50-60° C., a sample was taken and in-process control of the reaction was performed until the reaction was completed. The reaction solution was cooled to 10-20° C., adjusted the pH of the reaction solution to 4-4.5 with 1N hydrochloric acid, extracted with ethyl acetate. The organic phase was washed with a freshly prepared L-cysteine sodium bicarbonate aqueous solution (L-cysteine:sodium bicarbonate:purified water=1:1.5:17.3; mass ratio) for 5 times, each wash was stirred for 1-2 hours, then the organic phase was washed with saturated brine aqueous solution, separated, dried with anhydrous sodium sulfate, filtered, washed with ethyl acetate, concentrated under reduced pressure until a large amount of solids were precipitated, filtered. The filter cake was washed with ethyl acetate, and dried under vacuum to obtain 14 g of the compound 2 with a yield of 80%.

The compound 2 can also be synthesized by one-pot method,

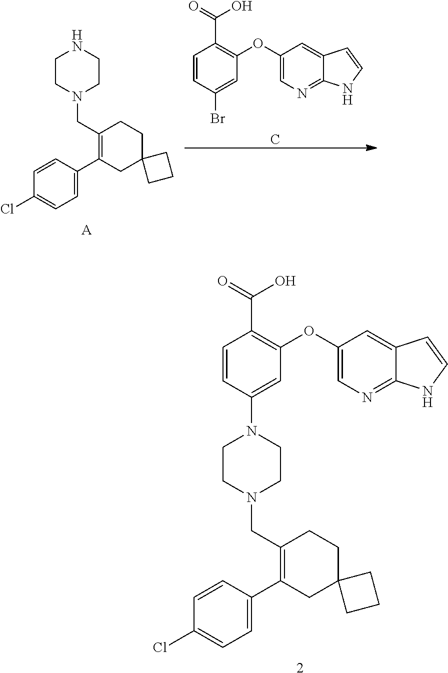

Toluene (4.9 g) and tetrahydrofuran (2.4 g) were added to a reactor, then the compound A (0.7 g) and the compound C (0.9 g) were added, bis(di-tert-butyl(4-(dimethylaminophenyl)phosphine)dichloropalladium(II) (0.08 g) and sodium tert-pentoxide (2.2 g) were added under the protection of nitrogen. The reaction solution was heated to 60° C. and stirred for 20 hours. After the reaction was completed, the reaction solution was cooled to room temperature, and adjusted the pH to 5.0-5.5 with 1N hydrochloric acid, then added with ethyl acetate to extract, separated, the organic phase was stirred with N-acetyl-L-cysteine sodium bicarbonate solution (N-acetyl-L-cysteine:sodium bicarbonate:water=1:1.1:12.8 mass ratio) for 3 times, separated, the organic phase was dried over anhydrous sodium sulfate, filtered, and concentrated under reduced pressure to obtain a crude product. The crude product was purified by column chromatography (mobile phase:dichloromethane:methanol=20:1), 0.72 g of the compound 2 was obtained with a yield of 47%.

¹HNMR (400 MHz, d-DMSO): δ ppm: 11.62 (1H, s), 7.99 (1H, d, J=2.4), 7.75 (1H, d, J=8.8), 7.46 (1H, t, J₁=6.0, J₂=2.8), 7.39 (1H, d, J=2.8), 7.33 (2H, J=2.8), 7.05 (2H, d, J=8.0), 6.70 (1H, dd, J₁=8.8, J₂=2.0), 6.35 (1H, t, J₁=4.4, J₂=2.4), 6.33 (1H, d, J=1.6), 3.08 (4H, m), 2.68 (2H, s), 2.15-2.23 (8H, m), 1.67-1.87 (6H, m), 1.58-1.61 (2H, m).

Step 3 Synthesis of the Compound 3

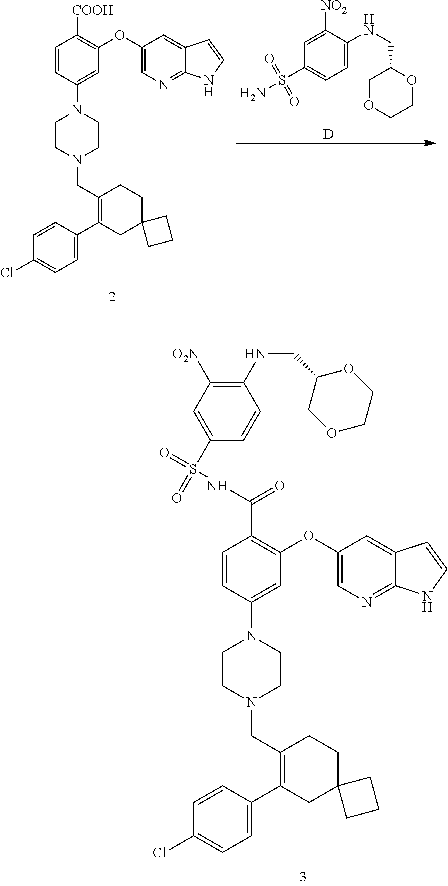

The compound D (2.6 g, 0.008 mol), DMAP (2 g, 0.016 mol) and NMP (15 mL) were added to a reactor. The reaction system was stirred at room temperature for 0.5 hour until the system was clear, added with EDC.HCl (2.2 g), added with a mixed solution of the compound 2 (compound 2+triethylamine+dichloromethane=5 g (0.009 mol)+1.6 g+75 mL). The reaction mixture was stirred overnight, added with N,N-dimethylethylenediamine (1.8 g) to quench the reaction, stirred at room temperature for 4-5 hours, washed with water and 10% acetic acid aqueous solution successively, and adjusted the pH to about 7 with 6% sodium bicarbonate aqueous solution, the organic phase was concentrated and added with 1,4-dioxane until the solution was clear, stirred for 0.5 hour, crystallized under natural cooling, stirred at room temperature overnight, and filtered to obtain 7 g of a crude product.

Recrystallization method of the compound 3: at room temperature, dichloromethane (30 mL) was added to dissolve the crude product, added with 1,4-dioxane (95 mL) dropwise in 0.5 hour, stirred for 1 hour, concentrated, and cooled naturally to 20° C., filtered and washed the filter cake with 1,4-dioxane to obtain 6 g of the compound 3 with a yield of 78%.

¹HNMR (400 MHz, d-DMSO): δ ppm 11.70 (1H, s), 11.35 (1H, br), 8.59 (2H, m), 8.05 (1H, d, J=2.6), 7.84 (1H, dd, J₁=9.2, J₂=2.3), 7.51 (3H, m), 7.33 (2H, d, J=8.4), 7.10 (1H, d, J=9.2), 7.04 (2H, d, J=8.4), 6.66 (1H, dd, J₁=8.8, J₂=1.2), 6.39 (1H, dd, J₁=3.6, J₂=2.0), 6.19 (1H, d, J=1.2), 3.77-3.82 (3H, m), 3.64 (1H, t, J=11.2), 3.62 (1H, dd, J₁=10.8, J₂=2.4), 3.30-3.52 (4H, m), 3.06 (4H, m), 2.72 (2H, m), 2.13-2.23 (8H, m), 1.67-1.86 (6H, m), 1.58 (2H, m).

1. A method for synthesizing compound 1, comprising conducting a Buchwald-Hartwig coupling reaction as shown below with compound A and compound B in a solvent and in the presence of a base and a palladium catalyst to obtain compound 1; 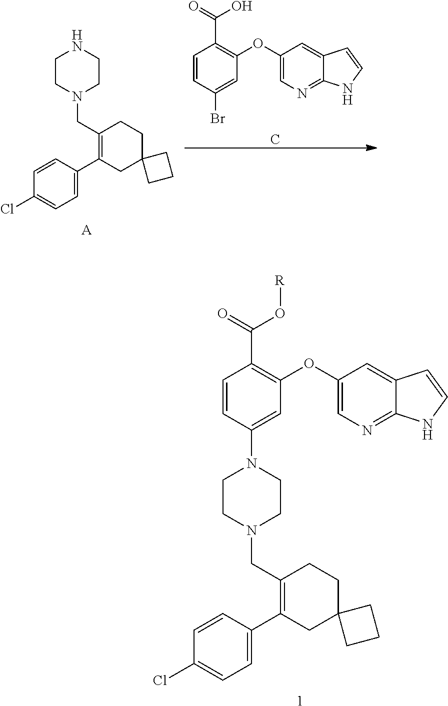
wherein R is C₁-C₈ alkyl.

2. The method of claim 1, wherein
the palladium catalyst is selected from: palladium acetate, [1,1′-bis(diphenylphosphino) ferrocene] dichloro palladium(II), tetrakis (triphenylphosphine)palladium, bis(triphenylphosphine) palladium(II) chloride, palladium on carbon, palladium hydroxide, [1,3-bis(2,6-diisopropylphenyl) imidazol-2(3H)-ylidene](3-chloro-1-pyridyl)palladium(IV) chloride, tris(dibenzylideneacetone) dipalladium, bis(di-tert-butyl(4-(dimethylaminophenyl) phosphine)dichloropalladium(II), and mixtures thereof;
the base is an inorganic base, an organic base, or mixtures thereof, wherein
the inorganic base is an alkali metal hydroxide, an alkali metal carbonate, an alkali metal phosphate, an alkali metal bicarbonate or a mixture thereof, and
the organic base is R^{m1}OM1, (M2)N(Rⁿ¹Rⁿ²) or a mixture thereof, wherein each of M1 and M2 is an alkali metal; each of Rⁿ¹ and Rⁿ² is C₁-C₄ alkyl or —Si(R^{s1}R^{s2}R^{s3}); and each of R^{m1}, R^{s1}, R^{s2} and R^{s3} is C₁-C₄ alkyl;
a molar ratio of compound A to the base is 1:(3-10);
a molar ratio of compound A to the palladium catalyst is 1:(0.01-0.5);
a molar ratio of compound A to compound B is 1:(1-3);
the solvent is a chlorinated alkane solvent, an aromatic hydrocarbon solvent, an ether solvent or a mixture thereof, wherein
the aromatic hydrocarbon solvent is benzene, toluene, xylene, chlorobenzene or a mixture thereof;
the ether solvent is diethyl ether, tetrahydrofuran, 2-methyl tetrahydrofuran, 1,4-dioxane, methyl tert-butyl ether, ethylene glycol dimethyl ether or a mixture thereof;
the temperature of the Buchwald Hartwig coupling reaction is 40-100° C.;
the Buchwald-Hartwig coupling reaction is conducted under the protection of a gas, wherein the gas in the gas protection is nitrogen, helium or argon;
the Buchwald-Hartwig coupling reaction is conducted in the presence of a ligand or in the absence of a ligand.

3. The method of claim 1, wherein;
the palladium catalyst is bis(di-tert-butyl(4-(dimethylaminophenyl phosphine) dichloropalladium(II);
the base is sodium hydroxide, potassium hydroxide, potassium carbonate, sodium carbonate, potassium phosphate, lithium carbonate, cesium carbonate, potassium tert-butoxide, sodium tert-butoxide, n-butyllithium, KHMDS, NaHMDS, LDA, potassium tert-pentoxide, sodium tert-pentoxide or a mixture thereof;
a molar ratio of compound A to the base is 1:(4.5-8);
a molar ratio of compound A to the palladium catalyst is 1:(0.05-0.2);
a molar ratio of compound A to compound B is 1:(1-1.5);
the solvent is a mixed solvent of an aromatic hydrocarbon solvent and an ether solvent, wherein a mass ratio of the aromatic hydrocarbon solvent to the ether solvent is 1:1-10:1;
the temperature of the Buchwald Hartwig coupling reaction is 40-70° C.;
when the Buchwald-Hartwig coupling reaction is conducted in the presence of the ligand, the ligand is selected from a monodentate phosphine ligand, a polydentate phosphine ligand or a mixture thereof;
when the Buchwald-Hartwig coupling reaction is conducted in the presence of the ligand, a molar ratio of compound A to the ligand is 1:(0.01-0.5).

4. The method of claim 1, wherein;
a salt of compound A is used to conduct the Buchwald-Hartwig coupling reaction;
the salt of compound A is a salt formed by compound A and an acid;
the acid is an inorganic acid or an organic acid;
the inorganic acid is hydrochloric acid, sulfuric acid or phosphoric acid;
the organic acid is trifluoroacetic acid.

5. The method of claim 4, wherein
the salt of compound A dissociates to form compound A

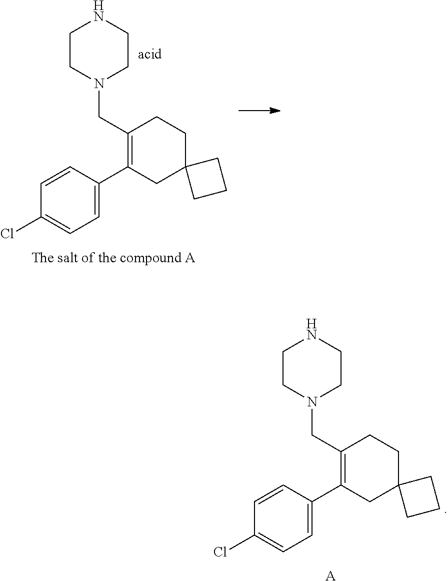

6. The method of claim 5, wherein
the base is an alkali metal carbonate, an alkali metal bicarbonate, an alkali metal phosphate, an alkali metal hydrogen phosphate or a mixture thereof;
an amount of the base renders the pH value in the reaction solution between 8 and 9;
the solvent is a mixed solvent of water and an organic solvent, wherein the organic solvent is a chlorinated alkane solvent, an aromatic hydrocarbon solvent or a mixture thereof; the chlorinated alkane solvent is dichloromethane, chloroform, 1,2-dichloroethane or a mixture thereof; the aromatic hydrocarbon solvent is benzene, toluene, xylene, chlorobenzene or a mixture thereof; a mass ratio of water to the organic solvent is 1:1-1:20.

7. The method in claim 5 further comprising conducting a reaction as shown below with compound A-7 in a solvent and in the presence of an acid to obtain the salt of compound A;

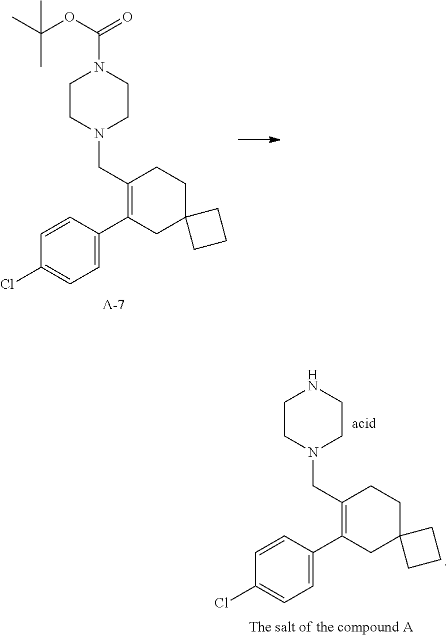

8. The method of claim 7, wherein
the acid is an inorganic acid or an organic acid, wherein the inorganic acid is hydrochloric acid, sulfuric acid or phosphoric acid; the organic acid is trifluoroacetic acid;
a mass ratio of compound A-7 to the acid is 1:(1-10);
the solvent is water, a C₁-C₆ alcohol solvent, a chlorinated alkane solvent, an ether solvent, an ester solvent or a mixture thereof, wherein the C₁-C₆ alcohol solvent is methanol, ethanol, isopropanol, tert-butanol, n-butanol or a mixture thereof; the chlorinated alkane solvent is dichloromethane, chloroform, 1,2-dichloroethane or a mixture thereof; the ether solvent is diethyl ether, tetrahydrofuran,
2-methyltetrahydrofuran, 1,4-dioxane, methyl tert-butyl ether, ethylene glycol dimethyl ether or a mixture thereof;
a mass ratio of compound A-7 to the solvent is 1:(1-10);
the reaction temperature is 30-100° C.

9. The method of claim 7, further comprising conducting a Borch reduction as shown below with compound A-6 and 1-Boc-piperazine in a solvent and in the presence of a reducing agent to obtain compound A-7;

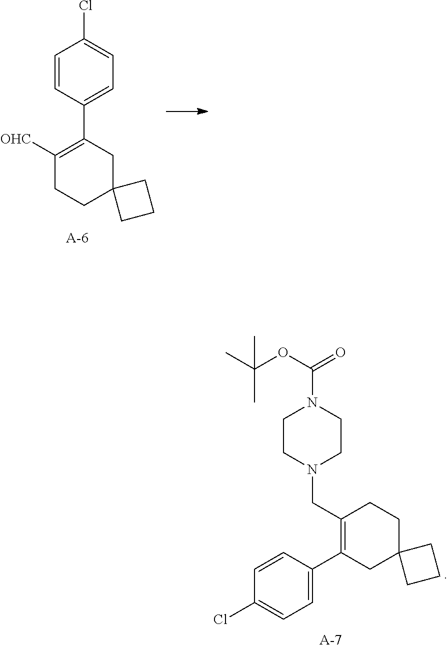

10. The method of claim 9, wherein,
the reducing agent is a metal borohydride;
a molar ratio of compound A-6 to the reducing agent is 1:(1-10), preferably 1:(3-10);
the solvent is a chlorinated alkane solvent, an ether solvent, a nitrile solvent, an ester solvent or a mixture thereof, wherein the chlorinated alkane solvent is dichloromethane, chloroform, 1,2-dichloroethane or a mixture thereof; the ether solvent is diethyl ether, tetrahydrofuran, 2-methyltetrahydrofuran, methyl tert-butyl ether or a mixture thereof; the nitrile solvent is acetonitrile; the ester solvent is ethyl acetate, isopropyl acetate or a mixture thereof;
a molar ratio of compound A-6 to 1-Boc-piperazine is 1:(1-5);
the temperature of the Borch reduction is room temperature to 50° C.;
is the reduction is conducted under the protection of a gas, wherein the gas in the gas protection is nitrogen, helium or argon.

11. The method of claim 9, further comprising conducting a coupling reaction as shown below with compound A-5 and 4-chloro phenylboronic acid in a solvent and in the presence of a palladium catalyst and a base to obtain compound A-6 under the protection of a gas;

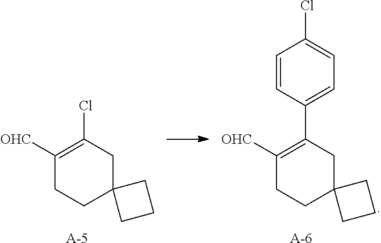

12. The method of claim 11, wherein,
the gas in the gas protection is nitrogen, helium or argon;
the palladium catalyst is palladium acetate, [1,1′-bis(diphenylphosphino)ferrocene]dichloro palladium(II), tetrakis (triphenylphosphine)palladium, bis(triphenylphosphine) palladium(II) chloride, palladium on carbon, palladium hydroxide, [1,3-bis(2,6-diisopropylphenyl)imidazol-2(31H)-ylidene](3-chloro-1-pyridyl)palladium(IV) chloride, tris(dibenzylideneacetone) dipalladium, bis(di-tert-butyl(4-(dimethylaminophenyl)phosphine)dichloropalladium(II), or a mixture thereof;
a molar ratio of compound A-5 to the palladium catalyst is 1:(0.001-0.05);
a molar ratio of compound A-5 to 4-chlorophenylboric acid is 1:(0.8-2.5);
the base is an inorganic base, wherein the inorganic base is an alkali metal carbonate;
a molar ratio of compound A-5 to the base is 1:(1-5);
the solvent is water, a C₁-C₆ alcohol solvent, an ether solvent or a mixture thereof, wherein for the mixed solvent, a mass ratio of the ether solvent to water and the alcohol solvent is (1-50):(1-50):1; the C₁-C₆ alcohol solvent is methanol, ethanol, isopropanol, tert-butanol, n-butanol or a mixture thereof; the ether solvent is diethyl ether, tetrahydrofuran, 2-methyltetrahydrofuran, 1,4-dioxane, methyl tert-butyl ether, ethylene glycol dimethyl ether or a mixture thereof;
the temperature of the coupling reaction is 30-70° C.;
the reaction is conducted in the presence of a fluorine-containing additive, wherein the fluorine-containing additive is tetrabutylammonium fluoride, cesium fluoride, potassium fluoride or a mixture thereof; a molar ratio of tie-compound A-5 to the fluorine-containing additive is 1:(0.1-1).

13. The method of claim 11, further comprising conducting a formylation reaction as shown below with compound A-4 in the presence of DMF and POCl₃ to obtain compound A-5;

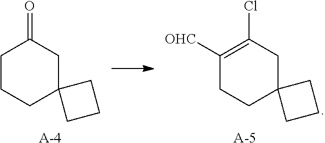

14. The method of claim 13, wherein
a molar ratio of compound A-4 to POCl₃ is 1:(1-5);
a molar ratio of compound A-4 to DMF is 1:(1-5);
the solvent is a chlorinated alkane solvent or a mixture thereof;
the temperature of the formylation reaction is the reflux temperature of the solvent under normal pressure.

15. The method of claim 13, further comprising conducting a reduction reaction as shown below with compound A-3 in a solvent and in the presence of an organic acid, hydrogen and a metal catalyst to obtain compound A-4;

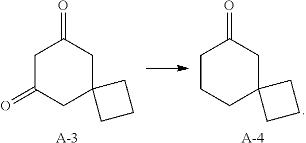

16. The method of claim 15, wherein;
the organic acid is methanesulfonic acid, p-toluenesulfonic acid, acetic acid or a mixture thereof;
a molar ratio of compound A-3 to the organic acid is 1:(0.01-0.5);
the metal catalyst is palladium, platinum, palladium on carbon, palladium acetate, palladium hydroxide or a mixture thereof;
a mass ratio of compound A-3 to the metal catalyst is 1:(0.01-0.1);
the solvent is water, a C₁-C₆ alcohol solvent or a mixture thereof, wherein the C₁-C₆ alcohol solvent is methanol, ethanol, isopropanol, tert-butanol, n-butanol or a mixture thereof;
the temperature of the reduction reaction is 50-100° C.;
the pressure of hydrogen is 0.5-0.6 MPa.

17. The method of claim 1, further comprising conducting a nucleophilic substitution reaction as shown below with compound B-1 and 5-hydroxy-7-azaindole in a solvent and in the presence of a base to obtain compound B; 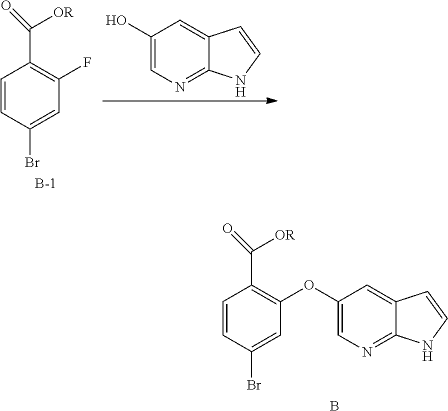
wherein R is C₁-C₈ alkyl.

18. The method of claim 17, wherein
the base is an inorganic base, an organic base or a mixture thereof, wherein the inorganic base is potassium phosphate, sodium carbonate, sodium bicarbonate, potassium carbonate, sodium hydride or a mixture thereof; the organic base is potassium tert-butoxide, sodium tert-butoxide or a mixture thereof;
a molar ratio of compound B-1 to the base is 1:(1-5);
the solvent is a nitrile solvent, an amide solvent or a mixture thereof;
a molar ratio of compound B-1 to 5-hydroxy-7-azaindole is 1:(1-5);
the temperature of the nucleophilic substitution reaction is 50-100° C.

19. The method of claim 17, further comprising conducting an esterification reaction as shown below with 2-fluoro-4-bromobenzoic acid and alcohol ROH in a solvent and in the presence of a condensing agent to obtain compound B-1; 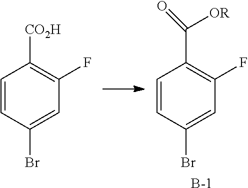
wherein R is C₁-C₈ alkyl.

20. The method of claim 19, wherein;
the condensing agent is EDC.HCl, CDI, DCC, HOBt, HOAT or a mixture thereof;
a molar ratio of 2-fluoro-4-bromobenzoic acid to the condensing agent is 1:(1-5);
the solvent is a chlorinated alkane solvent;
a molar ratio of 2-fluoro-4-bromobenzoic acid to the alcohol is 1:(1-10);
the temperature of the esterification reaction is room temperature to 50° C.

21. A method for synthesizing compound 2, which refers to either method 1 or method 2:
method 1 comprises conducting a hydrolysis reaction as shown below with compound 1 in a solvent and in the presence of an acid or a base to obtain compound 2; 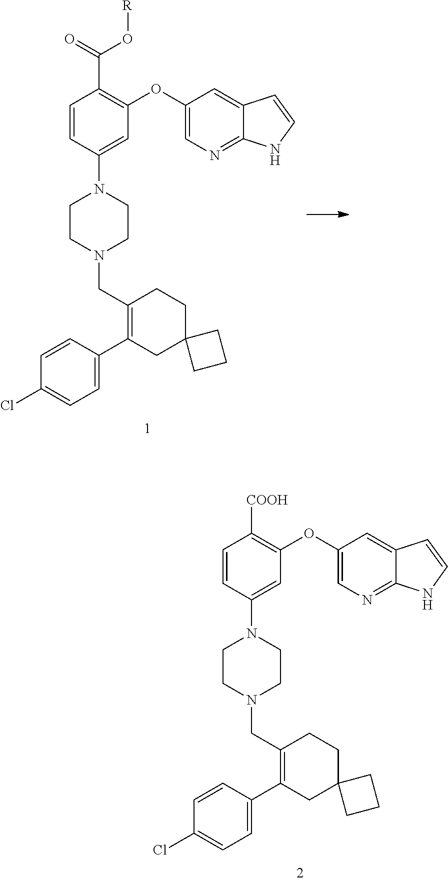
wherein R is C₁-C₈ alkyl;
method 2 comprises conducting a Buchwald-Hartwig coupling reaction as shown below with compound C and compound A in a solvent and in the presence of a base and a palladium catalyst to obtain compound 2;

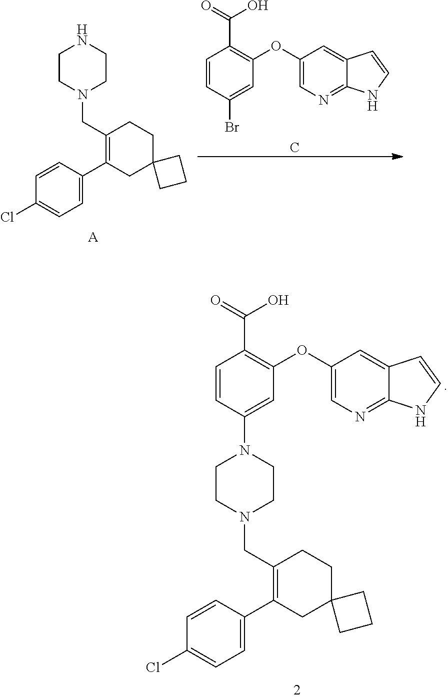

22. The method of claim 21, wherein
in the method 1, the acid is an inorganic acid, an organic acid or a mixture thereof, wherein the inorganic acid is hydrochloric acid, sulfuric acid or a mixture thereof; the organic acid is acetic acid, trifluoroacetic acid or a mixture thereof; a molar ratio of the acid to compound A is 1:(0.5-1);
in the method 1, the base is an inorganic base, an organic base or a mixture thereof, wherein; the inorganic base is an alkali metal hydroxide, an alkali metal carbonate, an alkali metal bicarbonate or a mixture thereof; the organic base is triethylamine, pyridine, DBU, DIPEA, triethylenediamine, DBN, DMAP, N-methylmorpholine, tetramethylethylenediamine, potassium tert-butoxide, tert-butanol sodium, n-butyllithium, KHMDS, NaHMDS, LDA, potassium tert-pentoxide, sodium tert-pentoxide or a mixture thereof; a molar ratio of the base to compound A is 1:(0.5-1);
in the method 1, the solvent is water, an ether solvent or a mixture thereof, wherein; the ether solvent is diethyl ether, tetrahydrofuran, 2-methyltetrahydrofuran, 1,4-dioxane, methyl tert-butyl ether or a mixture thereof;
in the method 1, the temperature of the hydrolysis reaction is room temperature to 60° C.

23. A method for synthesizing compound 3, comprising conducting an amidation reaction as shown below with compound 2 and compound D in the presence of a condensing agent, a base and a catalyst to obtain compound 3;

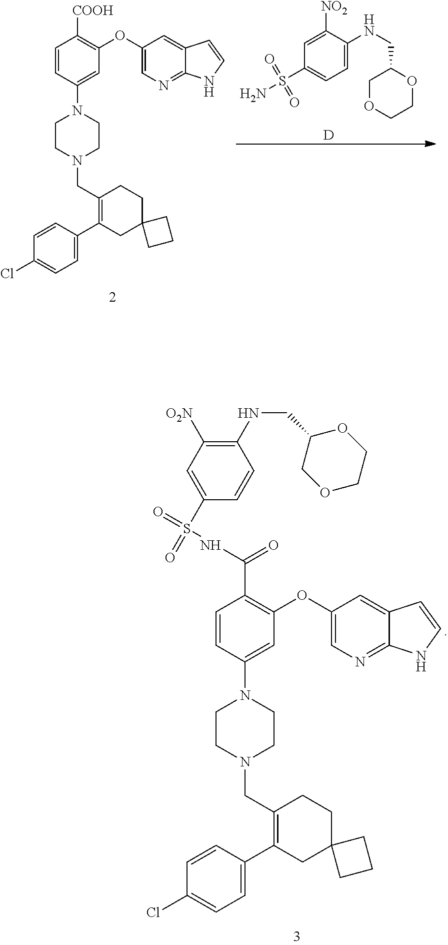

24. The method of claim 23, wherein
the condensing agent is DCC, EDC.HCl, CDI, HATU, HBTU, TBTU, HOBT, HOAT or a mixture thereof;
a molar ratio of compound 2 to the condensing agent is 1:(1-2);
the base is an inorganic base, an organic base or a mixture thereof; wherein the inorganic base is an alkali metal hydroxide, an alkali metal carbonate, an alkali metal bicarbonate or a mixture thereof; the organic base is triethylamine, N,N-diisopropylethylamine, pyridine or a mixture thereof;
a molar ratio of compound 2 to the base is 1:(1-5);
the catalyst is DMAP;
a mass ratio of compound 2 to the catalyst is 1:(0.1-1);
a molar ratio of compound 2 to compound D is 1:(0.8-1.5);
the temperature of the amidation reaction is 20-50° C.;

25. The method claim 23, further comprising conducting a reaction as shown below with compound D-1 and (S)-2-aminomethyl-1,4-dioxane hydrochloride in a solvent and in the presence of a base to obtain compound D; 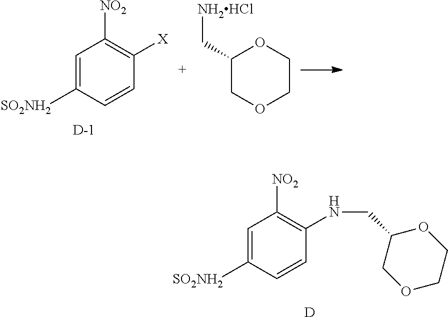
wherein X is halogen.

26. The method of claim 25, wherein;
the solvent is a nitrile solvent;
the base is an inorganic base, an organic base or a mixture thereof, wherein the inorganic base is an alkali metal hydroxide, an alkali metal carbonate or a mixture thereof;
a molar ratio of compound D-1 to the base is 1:(1-10);
a molar ratio of compound D-1 to (S)-2-aminomethyl-1,4-dioxane hydrochloride is 1:(1-2);
the reaction temperature is room temperature to the solvent reflux temperature under normal pressure.

27. A method for synthesizing the salt of compound A, comprising conducting a reaction as shown below with compound A-7 in a solvent and in the presence of an acid to obtain the salt of compound A;

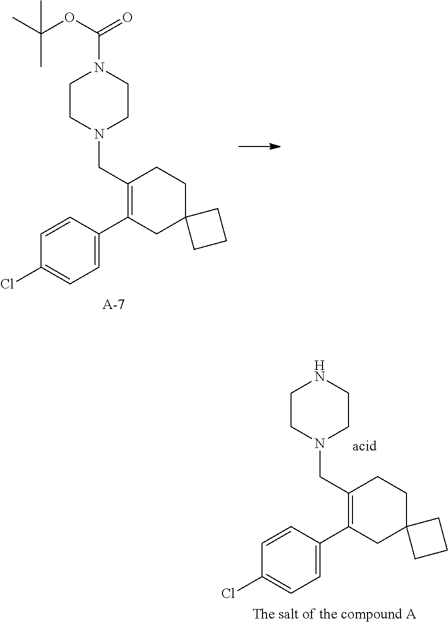

28. A method for synthesizing compound B, comprising conducting a nucleophilic substitution reaction as shown below with compound B-1 and 5-hydroxy-7-azaindole in the presence of a solvent and a base to obtain compound B; 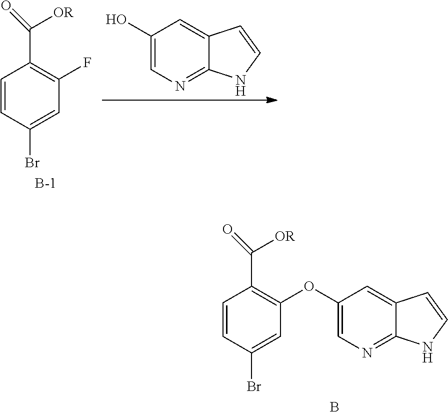
wherein R is C₁-C₈ alkyl;
the conditions of the method for synthesizing the salt of the compound B are the same as those defined in claim 18.

29. A method for synthesizing compound D, comprising conducting a reaction as shown below with compound D-1 and (S)-2-aminomethyl-1,4-dioxane hydrochloride in a solvent and in the presence of a base to obtain compound D; 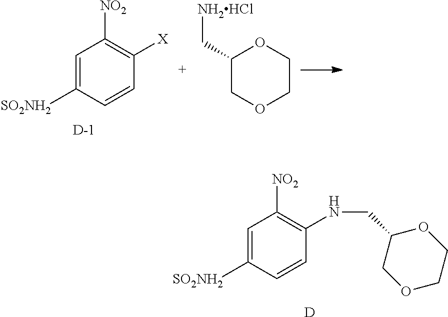
wherein X is halogen;
the conditions of the method for synthesizing the salt of the compound D are the same as those defined in claim 24.

30. A compound or a pharmaceutically acceptable salts thereof selected from one of the following: 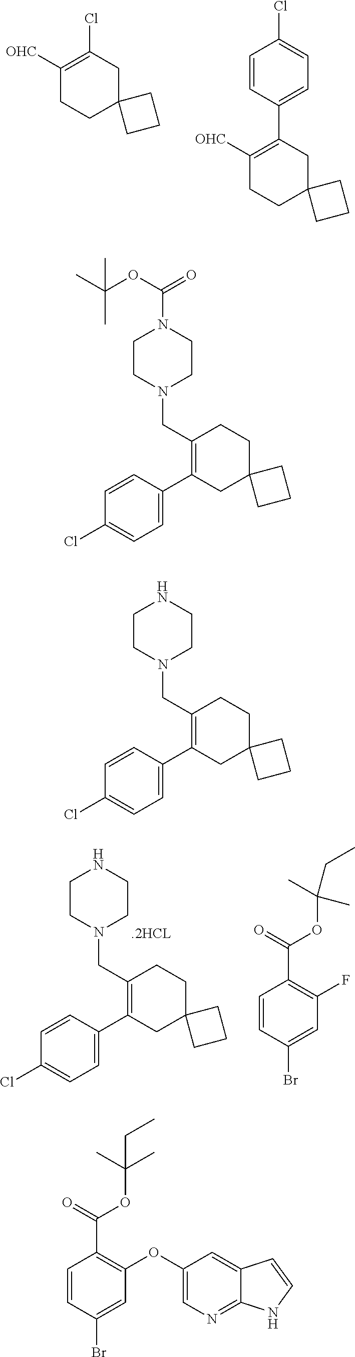 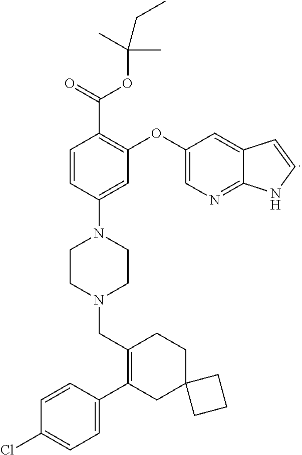 [SMILES: CCC(C)(C)OC(=O)c1ccc(N2CCN(CC3=C(c4ccc(Cl)cc4)CC4(CCC4)CC3)CC2)cc1Oc1cnc2[nH]ccc2c1]
